SEQUENCE LISTING (53 sequences)

SEQ ID NO: 1 - DNA, 21 bp, Artificial Sequence
  Chemically Synthesized
cccacauacucauguaguatt

SEQ ID NO: 2 - DNA, 21 bp, Artificial Sequence
  Chemically Synthesized
cuuaacgucucacaauugatt

SEQ ID NO: 3 - DNA, 21 bp, Artificial Sequence
  Chemically Synthesized
gccuuauagagguaauacatt

SEQ ID NO: 4 - DNA, 21 bp, Artificial Sequence
  Chemically Synthesized
cucaaugccuugaaacgaatt

SEQ ID NO: 5 - DNA, 21 bp, Artificial Sequence
  Chemically Synthesized
gagaugaaccuccagucuutt

SEQ ID NO: 6 - RNA, 19 nt, Artificial Sequence
  Chemically Synthesized
gagaccuaccuccggauca

SEQ ID NO: 7 - DNA, 21 bp, Artificial Sequence
  Chemically Synthesized
cguacgcggaauacuucgatt

SEQ ID NO: 8 - DNA, 21 bp, Artificial Sequence
  Chemically Synthesized
cugugugguguguacuuuatt

SEQ ID NO: 9 - DNA, 21 bp, Artificial Sequence
  Chemically Synthesized
ccuacaaucaguucagaautt

SEQ ID NO: 10 - DNA, 21 bp, Artificial Sequence
  Chemically Synthesized
cagauuuguccuucccauutt

SEQ ID NO: 11 - DNA, 24 bp, Artificial Sequence
  Chemically Synthesized
ggctaactagggaacccactgctt

SEQ ID NO: 12 - DNA, 17 bp, Artificial Sequence
  Chemically Synthesized
agccgagtcctgcgtcg

SEQ ID NO: 13 - DNA, 30 bp, Artificial Sequence
  Chemically Synthesized
cctcaataaagcttgccttgagtgcttcaa

SEQ ID NO: 14 - DNA, 23 bp, Artificial Sequence
  Chemically Synthesized
aactagggaacccactgcttaag

SEQ ID NO: 15 - DNA, 21 bp, Artificial Sequence
  Chemically Synthesized
tgctgggattacaggcgtgag

SEQ ID NO: 16 - DNA, 30 bp, Artificial Sequence
  Chemically Synthesized
acactacttgaagcactcaaggcaagcttt

SEQ ID NO: 17 - DNA, 21 bp, Artificial Sequence
  Chemically Synthesized
tccgctaccataatcatcgct

SEQ ID NO: 18 - DNA, 18 bp, Artificial Sequence
  Chemically Synthesized
ccgtggagtgtggcgagt

SEQ ID NO: 19 - DNA, 26 bp, Artificial Sequence
  Chemically Synthesized
tccccaccggcgtcaaagtatttagc

SEQ ID NO: 20 - DNA, 21 bp, Artificial Sequence
  Chemically Synthesized
ugaugaacguucuauguautt

SEQ ID NO: 21 - DNA, 21 bp, Artificial Sequence
  Chemically Synthesized
cuauguauuucaucggauatt

SEQ ID NO: 22 - DNA, 21 bp, Artificial Sequence
  Chemically Synthesized
ggauuaugcgacacaauaatt

SEQ ID NO: 23 - DNA, 21 bp, Artificial Sequence
  Chemically Synthesized
guggucaacuugauugagatt

SEQ ID NO: 24 - DNA, 21 bp, Artificial Sequence
  Chemically Synthesized
gugaugggaugauugcugatt

SEQ ID NO: 25 - DNA, 21 bp, Artificial Sequence
  Chemically Synthesized
gcugacauaucaaguauautt

SEQ ID NO: 26 - DNA, 21 bp, Artificial Sequence
  Chemically Synthesized
cucauaccaucuauucagatt

SEQ ID NO: 27 - DNA, 21 bp, Artificial Sequence
  Chemically Synthesized
cucacaucaagcugacauatt

SEQ ID NO: 28 - DNA, 21 bp, Artificial Sequence
  Chemically Synthesized
cacaccuucucucacaucatt

SEQ ID NO: 29 - DNA, 21 bp, Artificial Sequence
  Chemically Synthesized
gcuguuuccaccgccaugatt

SEQ ID NO: 30 - DNA, 21 bp, Artificial Sequence
  Chemically Synthesized
ugagaauccuguuguuagutt

SEQ ID NO: 31 - DNA, 21 bp, Artificial Sequence
  Chemically Synthesized
ccugagaauccuguuguuatt

SEQ ID NO: 32 - DNA, 21 bp, Artificial Sequence
  Chemically Synthesized
gagaauccuguuguuaguatt

SEQ ID NO: 33 - DNA, 21 bp, Artificial Sequence
  Chemically Synthesized
cggaucauguucucagaaatt

SEQ ID NO: 34 - DNA, 21 bp, Artificial Sequence
  Chemically Synthesized
ggaaguggaugaguuccuutt

SEQ ID NO: 35 - DNA, 21 bp, Artificial Sequence
  Chemically Synthesized
ccaggugacuucuccagugtt

SEQ ID NO: 36 - DNA, 21 bp, Artificial Sequence
  Chemically Synthesized
caaucagagucuucuuugatt

SEQ ID NO: 37 - DNA, 21 bp, Artificial Sequence
  Chemically Synthesized
cucauacagcugacgggautt

SEQ ID NO: 38 - DNA, 21 bp, Artificial Sequence
  Chemically Synthesized
guuuacaacuugaacgauatt

SEQ ID NO: 39 - DNA, 21 bp, Artificial Sequence
  Chemically Synthesized
ggcagauagacacaguauatt

SEQ ID NO: 40 - DNA, 21 bp, Artificial Sequence
  Chemically Synthesized
cauacucuccuuuaccauatt

SEQ ID NO: 41 - DNA, 21 bp, Artificial Sequence
  Chemically Synthesized
ccuuuaccauacuacuggatt

SEQ ID NO: 42 - DNA, 21 bp, Artificial Sequence
  Chemically Synthesized
gaguggagcugaagcuaagtt

SEQ ID NO: 43 - DNA, 21 bp, Artificial Sequence
  Chemically Synthesized
cacuagaagaucucagaaatt

SEQ ID NO: 44 - DNA, 21 bp, Artificial Sequence
  Chemically Synthesized
cucgugaagagauguuacatt

SEQ ID NO: 45 - DNA, 21 bp, Artificial Sequence
  Chemically Synthesized
cuauuucacugggagugautt

SEQ ID NO: 46 - DNA, 21 bp, Artificial Sequence
  Chemically Synthesized
gcgaauugggcugaaauautt

SEQ ID NO: 47 - DNA, 21 bp, Artificial Sequence
  Chemically Synthesized
gguugauacccaaagaucatt

SEQ ID NO: 48 - DNA, 21 bp, Artificial Sequence
  Chemically Synthesized
cuguucccaagagaccuaatt

SEQ ID NO: 49 - DNA, 21 bp, Artificial Sequence
  Chemically Synthesized
guccuucuggucaccucautt

SEQ ID NO: 50 - DNA, 21 bp, Artificial Sequence
  Chemically Synthesized
gcuccgagcugcgagauaatt

SEQ ID NO: 51 - DNA, 21 bp, Artificial Sequence
  Chemically Synthesized
gcaagcacuucuuucuuuatt

SEQ ID NO: 52 - DNA, 21 bp, Artificial Sequence
  Chemically Synthesized
cgauacgucacagccuucatt

SEQ ID NO: 53 - DNA, 21 bp, Artificial Sequence
  Chemically Synthesized
cagucagaaggacaggacatt

(19) United States
(12) Patent Application Publication
(10) Pub. No.: US 2011/0065087 A1
(43) Pub. Date: Mar. 17, 2011

(54) NOVEL HIV TARGETS

(76) Inventors: Amy S. Espeseth, Chalfont, PA (US); Daria J. Hazuda, Doylestown, PA (US); Min Xu, Ambler, PA (US); Honglin Zhou, North Wales, PA (US)

(21) Appl. No.: 12/739,186

(22) PCT Filed: Oct. 20, 2008
(86) PCT No.: PCT/US08/11931

§ 371 Date: Apr. 22, 2010

Related U.S. Application Data
(60) Provisional application No. 61/000,165, filed on Oct. 24, 2007.

(51) Int. Cl. C12Q 1/70; C07H 21/00; C12Q 1/68

(52) U.S. Cl. 435/5; 536/24.5; 435/6

(57) ABSTRACT

A set of cellular genes that were identified by siRNA screening as being essential for Human Immunodeficiency Virus (HIV) infection. These genes are host cellular factors involved in DNA repair, specifically in the base excision repair pathway.

### BACKGROUND OF THE INVENTION

After the Human Immunodeficiency Virus (HIV) integrates into the host genome, a gap remains between the integrated viral DNA and the host chromosome. Because HIV integrase is incapable of repairing the gap, it has long been assumed that the damage is repaired by host DNA repair factors. A number of DNA repair-associated proteins have been linked to retroviral transduction, as it is known that host DNA repair pathways are required to complete the process of retroviral integration (Kilzer, et al., 2003; Daniel, et al., 2004; Parissi, et al., 2003; Mulder et al., 2002), and such information provides an indication that host cellular factors may be potential targets for antiviral therapy.

Past drug discovery programs for HIV have largely targeted viral enzymes, including reverse transcriptase, protease, and integrase, and compounds targeting these enzymes have become the standard treatment for HIV infection. Although anti-retroviral therapy successfully suppresses viral replication, latent viral reservoirs coupled with the poor fidelity of HIV reverse transcriptase often leads to resistance. Because the pharmacological targeting of required host factors may slow or prevent viral resistance, the identification of novel host factors as targets for HIV therapy may represent a significant advance for the field of HIV therapeutics. The base excision repair pathway of human DNA repair appears to provide several such host factors as indicated by results from knockdown screening using siRNAs.

Thus, there is an unmet need to identify novel targets for the treatment of HIV infection, which might include host cellular factors, particularly those involved in DNA repair, more particularly those of the base excision repair pathway.

### SUMMARY OF THE INVENTION

Several human genes encoding cellular proteins that serve in host DNA repair have been identified by siRNA screening as being essential for HIV infection. Knockdown of expression of these genes using siRNA decreases HIV transduction of P4/R5 HeLa cells in a single cycle HIV infectivity assay. These genes and the proteins encoded thereby thus potentially provide targets for evaluating the ability of compounds to inhibit HIV infection, which might include both compounds targeting the nucleic acids encoding the proteins identified and those targeting the proteins themselves.

Thus, in one embodiment of the present invention there is provided isolated host cellular proteins involved in HIV infection useful as research tools selected from the group consisting of: MUTYH; NEIL3; LIG3; POLB; XRCC1, or a protein substantially similar thereto.

“Substantially similar” is defined as a sequence identity of at least 95% to the target protein. Nucleic acid and protein substantially similar to a particular identified sequence provide sequences with a small number of changes to the particular identified sequence. Substantially similar sequences include sequences containing one or more naturally occurring polymorphisms or changes that are artificially produced. Each change is independently an addition, deletion or substitution. A substantially similar nucleic acid is at least 95% identical to a reference sequence. The substantially similar nucleic acid sequence should encode a protein that does not have significantly less activity than the protein encoded by the reference sequence.

In another embodiment of the invention, there is provided an assay for identifying a compound as an HIV inhibitor comprising the steps of: identifying a compound that downregulates or otherwise inhibits the activity or expression of a target protein that is a component of a DNA repair pathway of a human cell, specifically of the base excision repair pathway; and determining the ability of said compound to inhibit HIV. Said assay may be more particularly characterized in that the target protein is either or a protein having a sequence identity with one or more members selected from the group consisting of: MUTYH; NEIL3; LIG3; POLB; and XRCC1.

In another embodiment of the present invention there is provided a method of screening for a compound which down-regulates the expression of one or more components of a DNA repair pathway, specifically the base excision repair pathway, of a human cell, thereby decreasing HIV infection, comprising the steps of: contacting the one or more components of a DNA repair pathway of a human cell with a noncircularized HIV DNA in the presence of a test compound; contacting the or more components of a DNA repair pathway of a human cell with a noncircularized HIV DNA in the absence of a test compound; and determining the effect of the test compound on HIV integration as measured by the amount of circularization. More particularly, the one or more components of a DNA repair pathway of a human cell may be a nucleic acid molecule encoding a polypeptide selected from the group consisting of: MUTYH; NEIL3; LIG3; POLB; and XRCC1 and homologs thereof.

This invention additionally relates to compounds, compositions, and methods useful for modulating the expression of genes, such as those genes associated with viral infection (e.g. HIV-1, HIV-2), for example, human genes of the DNA repair pathway and related genes, using short interfering nucleic acid (siNA) molecules. Thus, in another embodiment of the present invention there is provided siNA molecules which act to downregulate expression of genes involved in DNA repair.

The terms “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of inhibiting or downregulating gene expression or viral replication by mediating RNA interference “RNAi” or gene silencing in a sequence-specific manner. These terms can refer to both individual nucleic acid molecules, a plurality of such nucleic acid molecules, or pools of such nucleic acid molecules. The siNA can be a double-stranded nucleic acid molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siNA can be assembled from two separate oligonucleotides, where one strand is the sense strand and the other is the antisense strand, wherein the antisense and sense strands are self-complementary. Alternatively, the siNA is assembled from a single oligonucleotide, where the self-complementary sense and antisense regions of the siNA are linked by means of a nucleic acid based or non-nucleic acid-based linker(s). The siNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. As used herein, siNA molecules need not be limited to those molecules containing only RNA, but further encompasses chemically-modified nucleotides and non-nucleotides. As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. Such siNA molecules are distinct from other nucleic acid technologies known in the art that mediate inhibition of gene expression, such as ribozymes, antisense, triplex forming, aptamer, 2,5-A chimera, or decoy oligonucleotides.

By “aptamer” or “nucleic acid aptamer” as used herein is meant a polynucleotide that binds specifically to a target molecule wherein the nucleic acid molecule has sequence that is distinct from sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art, see for example Gold et al., 1995, Ann Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628. Aptamer molecules of the invention can be chemically modified as is generally known in the art or as described herein.

This invention further relates to compounds, compositions, and methods useful for modulating such gene expression using short interfering nucleic acid (siNA) molecules. This invention also relates to compounds, compositions, and methods useful for modulating the expression and activity of other genes involved in DNA repair pathways and/or activity by RNA interference (RNAi) using small nucleic acid molecules.

By “RNA interference” or “RNAi” is meant a biological process of inhibiting or down regulating gene expression in a cell as is generally known in the art and which is mediated by short interfering nucleic acid molecules, see for example Zamore and Haley, 2005, Science, 309, 1519-1524; Vaughn and Martienssen, 2005, Science, 309, 1525-1526; Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). In addition, as used herein, the term RNAi is meant to be equivalent to other terms used to describe sequence specific RNA interference, such as post transcriptional gene silencing, translational inhibition, transcriptional inhibition, or epigenetics. For example, siNA molecules of the invention can be used to epigenetically silence genes at both the post-transcriptional level and the pre-transcriptional level. In a non-limiting example, epigenetic modulation of gene expression by siNA molecules of the invention can result from siNA mediated modification of chromatin structure or methylation patterns to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237). In another non-limiting example, modulation of gene expression by siNA molecules of the invention can result from siNA mediated cleavage of RNA (either coding or non-coding RNA) via RISC, or alternately, translational inhibition as is known in the art. In another embodiment, modulation of gene expression by siNA molecules of the invention can result from transcriptional inhibition (see for example Janowski et al., 2005, Nature Chemical Biology, 1, 216-222).

A siNA or RNAi inhibitor of the invention can be unmodified or chemically-modified. A siNA or RNAi inhibitor of the instant invention can be chemically synthesized, expressed from a vector, or enzymatically synthesized. The instant invention also features various chemically-modified synthetic short interfering nucleic acid (siNA) molecules capable of modulating target gene expression or activity in cells by RNA interference (RNAi). The instant invention also features various chemically-modified synthetic short nucleic acid (siNA) molecules capable of modulating RNAi activity in cells by interacting with miRNA, siRNA, or RISC, and hence down regulating or inhibiting RNA interference (RNAi), translational inhibition, or transcriptional silencing in a cell or organism. The use of chemically-modified siNA and/or RNAi inhibitors improves various properties of native siNA molecules and/or RNAi inhibitors through increased resistance to nuclease degradation in vivo and/or through improved cellular uptake.

By “RNA interference” or “RNAi” is meant a biological process of inhibiting or down regulating gene expression in a cell as is generally known in the art and which is mediated by short interfering nucleic acid molecules, see for example Zamore and Haley, 2005, Science, 309, 1519-1524; Vaughn and Martienssen, 2005, Science, 309, 1525-1526; Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). In addition, as used herein, the term RNAi is meant to be equivalent to other terms used to describe sequence specific RNA interference, such as post transcriptional gene silencing, translational inhibition, transcriptional inhibition, or epigenetics. For example, siNA molecules of the invention can be used to epigenetically silence genes at both the post-transcriptional level and the pre-transcriptional level. In a non-limiting example, epigenetic modulation of gene expression by siNA molecules of the invention can result from siNA mediated modification of chromatin structure or methylation patterns to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237). In another non-limiting example, modulation of gene expression by siNA molecules of the invention can result from siNA mediated cleavage of RNA (either coding or non-coding RNA) via RISC, or alternately, translational inhibition as is known in the art. In another embodiment, modulation of gene expression by siNA molecules of the invention can result from transcriptional inhibition (see for example Janowski et al., 2005, Nature Chemical Biology, 1, 216-222).

In one embodiment, the invention features one or more siNA molecules and methods that independently or in combination modulate the expression of genes encoding proteins of the DNA repair pathway, such as proteins selected from the group consisting of MUTYH; NEIL3; LIG3; POLB; and XRCC1.

The description below of the various aspects and embodiments of the invention is provided with reference to exemplary genes, including those encoding the proteins MUTYH; NEIL3; LIG3; POLB; and XRCC1. However, the various aspects and embodiments are also directed to other genes of the DNA repair pathway, in particular those in which downregulation has been shown to impact viral infections such as HIV-1 and HIV-2.

By “RNA interference” or “RNAi” is meant a biological process of inhibiting or down regulating gene expression in a cell as is generally known in the art and which is mediated by short interfering nucleic acid molecules, see for example Zamore and Haley, 2005, Science, 309, 1519-1524; Vaughn and Martienssen, 2005, Science, 309, 1525-1526; Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). In addition, as used herein, the term RNAi is meant to be equivalent to other terms used to describe sequence specific RNA interference, such as post transcriptional gene silencing, translational inhibition, transcriptional inhibition, or epigenetics. For example, siNA molecules of the invention can be used to epigenetically silence genes at both the post-transcriptional level and the pre-transcriptional level. In a non-limiting example, epigenetic modulation of gene expression by siNA molecules of the invention can result from siNA mediated modification of chromatin structure or methylation patterns to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237). In another non-limiting example, modulation of gene expression by siNA molecules of the invention can result from siNA mediated cleavage of RNA (either coding or non-coding RNA) via RISC, or alternately, translational inhibition as is known in the art. In another embodiment, modulation of gene expression by siNA molecules of the invention can result from transcriptional inhibition (see for example Janowski et al., 2005, Nature Chemical Biology, 1, 216-222).

In yet another embodiment, the invention features a siNA molecule that down-regulates expression of a target gene or that directs cleavage of a target RNA, for example, wherein the target gene or RNA comprises protein encoding sequence. In one embodiment, the invention features a siNA molecule that down-regulates expression of a target gene or that directs cleavage of a target RNA, for example, wherein the target gene or RNA comprises non-coding sequence or regulatory elements involved in target gene expression (e.g., non-coding RNA, miRNA, stRNA etc.). The siNA molecule may comprise an antisense region comprising a nucleotide sequence that is complementary to a nucleotide sequence encoding a protein or a portion thereof selected from the group consisting of MUTYH; NEIL3; LIG3; POLB; and XRCC1. The siNA molecule may further comprise a sense region, wherein said sense region comprises a nucleotide sequence of a gene or a portion thereof selected from the group consisting of mutyh; neil3; lig3; polb; and xrcc1.

In one embodiment, the sense region or sense strand of a siNA molecule of the invention is complementary to that portion of the antisense region or antisense strand of the siNA molecule that is complementary to a target polynucleotide sequence.

In one embodiment, the invention features one or more chemically-modified siNA constructs having specificity for MUTYH; NEIL3; LIG3; POLB; and XRCC1 target protein-expressing nucleic acid molecules, such as RNA encoding MUTYH; NEIL3; LIG3; POLB; and XRCC1 protein or non-coding RNA associated with the expression of MUTYH; NEIL3; LIG3; POLB; and XRCC1. Non-limiting examples of such chemical modifications include without limitation phosphorothioate internucleotide linkages, 2′-deoxyribonucleotides, 2′-O-methyl ribonucleotides, 2′-deoxy-2′-fluoro ribonucleotides, 4′-thio ribonucleotides, 2′-O-trifluoromethyl nucleotides, 2′-O-ethyl-trifluoromethoxy nucleotides, 2′-O-difluoromethoxy-ethoxy nucleotides (see, e.g., U.S. Ser. No. 10/981,966 filed Nov. 5, 2004, hereby incorporated by reference in its entirety), “universal base” nucleotides, “acyclic” nucleotides, 5-C-methyl nucleotides, 2′-deoxy-2′-fluoroarabino (FANA, see for example Dowler et al., 2006, Nucleic Acids Research, 34, 1669-1675) and terminal glyceryl and/or inverted deoxy-abasic residue incorporation. These chemical modifications, when used in various siNA constructs, (e.g., RNA based siNA constructs), are shown to preserve RNAi activity in cells while at the same time, dramatically increasing the serum stability of these compounds. In one embodiment, a siNA molecule is chemically modified at the internal positions of the siNA molecule. By “internal position” is meant the base paired positions of a siNA duplex. In one embodiment, a siNA molecule of the invention comprises modified nucleotides while maintaining the ability to mediate RNAi. The modified nucleotides can be used to improve in vitro or in vivo characteristics such as stability, activity, toxicity, immune response, and/or bioavailability.

It is further contemplated that a siNA molecule of the invention can comprise modified nucleotides as a percentage of the total number of nucleotides present in the siNA molecule. As such, a siNA molecule of the invention can generally comprise about 5% to about 100% modified nucleotides.

Additionally, a siNA molecule of the invention can comprise modified nucleotides at various other locations within the siNA molecule. E.g., it is contemplated that a double-stranded siNA molecule of the invention may comprise modified nucleotides at non-base paired or overhang regions of the siNA molecule. By “non-base paired” is meant, the nucleotides are not base paired between the sense strand or sense region and the antisense strand or antisense region or the siNA molecule. The overhang nucleotides can be complementary or base paired to a corresponding target polynucleotide sequence. It is further contemplated that a double stranded siNA molecule of the invention may comprise modified nucleotides at terminal positions of the siNA molecule. For example, such terminal regions include the 3′-position, 5′-position, for both 3′ and 5′-positions of the sense and/or antisense strand or region of the siNA molecule. A double stranded siNA molecule of the invention may additionally comprise modified nucleotides at base-paired or internal positions, non-base paired or overhang regions, and/or terminal regions, or any combination thereof.

In another embodiment, the double-stranded siNA molecule comprises one or more ribonucleotides. In one embodiment, one of the strands of the double-stranded siNA molecule comprises a nucleotide sequence that is complementary to a nucleotide sequence or a portion thereof of the mutyh; neil3; lig3; polb; and xrcc1 gene, and the second strand of the double-stranded siNA molecule comprises a nucleotide sequence substantially similar to the nucleotide sequence of the mutyh; neil3; lig3; polb; and xrcc1 gene, or a portion thereof.

In yet another embodiment of the present invention there is provided a siNA molecule which comprises blunt ends, i.e., ends that do not include any overhanging nucleotides. Any siNA molecule of the invention can comprise one or more blunt ends, i.e., where a blunt end does not have any overhanging nucleotides. By “blunt ends” is meant symmetric termini or termini of a double stranded siNA molecule having no overhanging nucleotides.

One embodiment of the invention provides an expression vector comprising a nucleic acid sequence encoding at least one siNA molecule of the invention in a manner that allows expression of the nucleic acid molecule. Another embodiment of the invention provides a mammalian cell comprising such an expression vector. The mammalian cell can be a human cell. The siNA molecule of the expression vector can comprise a sense region and an antisense region. The antisense region can comprise sequence complementary to a RNA or DNA sequence encoding a protein selected from the group consisting of MUTYH; NEIL3; LIG3; POLB; and XRCC1 and the sense region can comprise sequence complementary to the antisense region. The siNA molecule can comprise two distinct strands having complementary sense and antisense regions. The siNA molecule can comprise a single strand having complementary sense and antisense regions.

The present invention additionally features a chemically-modified short interfering nucleic acid molecule (siNA) capable of mediating RNA interference (RNAi) inside a cell or reconstituted in vitro system, wherein the chemical modification comprises a conjugate covalently attached to the chemically-modified siNA molecule. Non-limiting examples of conjugates contemplated by the invention include conjugates and ligands described in Vargeese et al., U.S. Ser. No. 10/427,160, filed Apr. 30, 2003, hereby incorporated by reference in its entirety. Said conjugate may be covalently attached to the chemically-modified siNA molecule via a biodegradable linker. Examples of specific conjugate molecules contemplated by the instant invention that can be attached to chemically-modified siNA molecules are described in Vargeese et al., U.S. Ser. No. 10/201,394, filed Jul. 22, 2002 and hereby incorporated by reference in its entirety.

The present invention further provides a method for modulating the expression of a gene within a cell comprising: (a) synthesizing a siNA molecule of the invention, which can be chemically-modified or unmodified, wherein one of the siNA strands comprises a sequence complementary to RNA of target sequence encoding a protein selected from the group consisting of MUTYH; NEIL3; LIG3; POLB; and XRCC1; and (b) introducing the siNA molecule into a cell under conditions suitable to modulate (e.g., inhibit) the expression of the target gene sequence selected from the group consisting of MUTYH; NEIL3; LIG3; POLB; and XRCC1, in the cell.

In one embodiment, the invention features a method for modulating the expression of a gene selected from the group consisting of mutyh; neil3; lig3; polb; and xrcc1 within a cell comprising: (a) synthesizing a siNA molecule of the invention, which can be chemically-modified or unmodified, wherein one of the siNA strands comprises a sequence complementary to RNA of a gene selected from the group consisting of mutyh; neil3; lig3; polb; and xrcc1 and wherein the sense strand sequence of the siNA comprises a sequence identical or substantially similar to the sequence of the target RNA; and (b) introducing the siNA molecule into a cell under conditions suitable to modulate (e.g., inhibit) the expression of the gene in the cell.

### BRIEF DESCRIPTION OF THE DRAWINGS

FIG. 1 illustrates tissue distribution for the novel target NEIL3.

FIG. 2 shows that MUTYH-targeting siRNAs knocked down MUTYH mRNA levels to less than 50% of wild-type levels.

FIG. 3 illustrates tissue distribution for the novel target MUTYH.

FIG. 4 depicts expression of MUTYH after HIV infection.

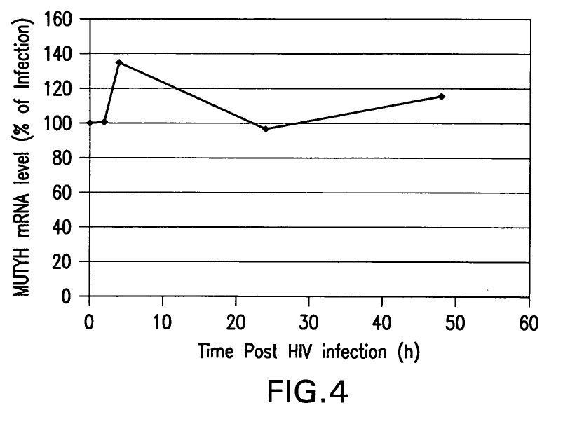

FIG. 5 shows that LIG3-targeting siRNAs knocked down LIG3 mRNA levels to less than 50% of wild type levels.

FIG. 6 illustrates tissue distribution for the novel target LIG3.

FIG. 7 depicts rescue of LIG3 siRNA-mediated inhibition of HIV replication following cotransfection of a siRNA with an expression vector for LIG3 cDNA that lacked the 3′-UTR.

FIG. 8 illustrates tissue distribution for the novel target POLB.

FIG. 9 illustrates tissue distribution for the novel target XRCC1.

### DETAILED DESCRIPTION OF THE INVENTION

Novel host cell protein targets for inhibiting HIV infection have been identified using siRNA screening. Because genes associated with the base excision repair pathway were noted as hits in the screen, additional siRNAs specifically targeting other base excision repair genes were tested. Five genes, MUTYH; NEIL3; LIG3; POLB; and XRCC1 were identified as novel targets. Such targets may prove useful not only for inhibiting HIV infection, but also for assessing the ability of compounds to inhibit HIV infection.

The present invention comprises a gene, MUTYH, which was identified by siRNA screening as being essential for HIV infection. Knockdown of expression of this gene using siRNA decreases HIV transduction of P4/R5 HeLa cells in HIV infectivity assays. Thus, inhibition of the function of MUTYH is a novel method for inhibition of HIV infection and represents a new target for drug discovery in HIV/AIDS.

The present invention further comprises a gene, NEIL3, which was identified by siRNA screening as being essential for Human Immunodeficiency Virus (HIV) infection. Knockdown of expression of this gene using siRNA decreases HIV transduction of P4/R5 HeLa cells in both single- and multiple-cycle HIV infectivity assays.

NEIL3 was identified in an unbiased siRNA screen, when a genome scale siRNA library targeting 18,670 genes was transfected into HeLaP4/R5 cells. Twenty-four hours following siRNA transfection, the cells were infected with HIV. Forty-eight or 96 hours after infection, the cells were assayed for expression of the β-gal reporter gene, as an indication that the virus had successfully integrated into the host genome and was producing sufficient quantities of the viral tat protein to induce expression through the LTR (Joyce et al., 2002). The life cycle of HIV is such that siRNAs that block expression of the reporter gene at 48 hours can be assumed to interfere with HIV infection at a point up to and including viral transcription, while siRNAs that block expression of the reporter gene at 96 hours may affect any point in the viral life cycle, including viral budding and release. SiRNAs that blocked or reduced the expression of β-gal were then examined in more detail.

Thus, inhibition of the function of NEIL3 is a novel method for inhibition of HIV infection and represents a new target for drug discovery in HIV/AIDS.

Additionally, the present invention comprises a gene, LIG3, a DNA ligase associated with the base excision repair pathway. LIG3 was shown to decrease HIV infectivity and is therefore another target for drug discovery.

The present invention further comprises a gene, POLB, a DNA polymerase associated with the base excision repair pathway, which was shown to inhibit HIV infectivity.

The present invention comprises a gene XRCC1, a DNA repair protein associated with the base excision repair pathway. XRCC1 also has been shown to inhibit HIV infectivity.

Inhibiting HIV infection by targeting host cellular factors has implications for both research and for antiviral therapy. Research applications of the present invention include providing methods to screen for compounds which inhibit HIV infection. Therapeutic applications include using identified compounds to treat or inhibit HIV infection.

### EXAMPLES

Examples are provided below further illustrating different features of the present invention and illustrate useful embodiments for practicing the invention. Theses embodiments should be viewed as exemplary of the present invention, rather than in any way limiting its scope.

### Example 1

### Inhibition of HIV Infection by NEIL3 siRNAs

The following protocol was used for all siRNA experiments:

Day 1: P4/R5 HeLa cells were plated at 500 cells per well into 384-well plates

Day 2: P4/R5 HeLa cells were transfected with siRNA pools as follows:
1. siRNAs were transfected in triplicate at a final concentration of 50 nM
2. Oligofectamine (Invitrogen) was added at a final concentration of 0.5%.
   Positive and negative control siRNAs were included as follows:
Cyclin T1 (positive control): purchased from Santa Cruz Biotechnology (cat #sc-35144)
CCNT1-1 (positive control): CCCACAUACUCAUGUAGUAdTdT (SEQ ID NO: 1)
CCNT1-4 (positive control): CUUAACGUCUCACAAUUGAdTdT (SEQ ID NO:2)
TSG101 (positive control): a pool of equal proportions of GCCUUAUAGAGGUAAUACAdTdT (SEQ ID NO:3), CUCAAUGCCUUGAAACGAAdTdT (SEQ ID NO: 4), and GAGAUGAACCUCCAGUCUUdTdT (SEQ ID NO: 5)
PLK1, a control for transfection efficiency, GAGACCUACCUCCGGAUCA (SEQ ID NO: 6)
Luciferase (negative control): CGUACGCGGAAUACUUCGAdTdT (SEQ ID NO: 7)
Mock (negative control): no siRNA

One column of mock-transfected cells with no virus added and one column with no cells plated were included on each plate as controls representing the bottom of the assay (lowest potential signal).
3. Oligofectamine was mixed with Optimem in a 1:50 ratio;
4. 20 μL of Optimem:Oligofectamine mixture was dispensed into each well of a sterile 384-well plate;
5. 1 μL of siRNA (10 μM) from each well of the siRNA stock plate was transferred into the Optimem/Oligofectamine-containing plates and was mixed up and down five times;
6. The plate was incubated at room temperature for 10 minutes; and
7. 5 μL of the siRNA-oligofectamine complex was added to each well of the HeLa(P4/R5) cells.

Day 3: Transfected HeLa(P4/R5) cells were infected with HXB2 HIV in the presence of an integrase inhibitor as follows:
1. Media was removed from the cells for two out of the three transfection plates;
2. The integrase inhibitor was diluted to 20 nM in media. 20 μL of the 20 nM solution was added to each well of two of the plates (the final concentration of the integrase inhibitor was equal to the IC50 of the compound for inhibition of viral infection in this assay (Anthony et al, 2004).
3. HXB2 HIV was diluted with media 1600× for plates to be assayed at 48 h (single cycle infection) and 6400× for plates to be assayed at 96 h (multiple cycle infection). 20 μL of diluted HXB2 was added to each well.
4. Viral infection was allowed to proceed for 48 h or 96 h, respectively.

Day 5 or 7 (48 or 96 hours after infection, respectively). Beta-galactosidase activity, which indicates viral infection, was measured as follows:
1. Media was removed from the cells;
2. Cells were washed with 40 μL PBS per well;
3. 20 μL of PBS was added to each well;
4. 20 μL of lysis buffer plus substrate (25:1) (Applied Biosystems, Cat# GSY10,000) were then added to each well and the plates were incubated at room temperature in the dark for 30 minutes; and
5. The plates were read using a VictorLight luminometer (PerkinElmer).

Day 5: The third transfected plate was assayed for siRNA-mediated cytotoxicity
1. Media was removed from all wells of the cell plate;
2. 40 μL Alamar Blue mixture (10% Alamar Blue Reagent, 2% FBS, Optimem) was added to each well of the plate;
3. Cells were incubated at 37° C., 5% CO₂ for 2 hrs; and
4. Fluorescence was read at 535 nm excitation (ex)/590 nm emission (em).

For data analysis, readings for each plate were normalized to the reading for the luciferase negative control and expressed as “% of Luciferase Control”. Hits were considered to be those siRNA pools that inhibited β-galactosidase activity by at least two-fold relative to luciferase. It was shown that the NEIL3 siRNA pool decreased β-galactosidase activity by 62% at 48 h and 69% at 96 h relative to luciferase.

The effective siRNA pool was an equal mixture of the following 3 siRNA duplexes (the targeted sequence is shown):

| | |
|---|---|
| CUGUGUGGUGUGUACUUUAdTdT | (SEQ ID NO: 8) |
| CCUACAAUCAGUUCAGAAUdTdT | (SEQ ID NO: 9) |
| CAGAUUUGUCCUUCCCAUUdTdT | (SEQ ID NO: 10) |

The siRNAs were synthesized by Sigma/Proligo and the siRNA design was done at Merck Research Laboratories using an algorithm from Rosetta.

### Example 2

### NEIL3 siRNAs are not Cytotoxic

Because NEIL3 siRNAs were identified via their inhibition of HIV infectivity, it is possible that these siRNAs appeared as hits in the infectivity screen simply due to cytotoxicity. For this reason, the NEIL3 siRNA pool was examined for cytotoxic effects in the cytotoxicity assay as described in Example 1. siRNAs that resulted in viability levels of less than 70%, as determined by Alamar Blue fluorescence, were considered to be cytotoxic. The NEIL3 siRNAs did not show any evidence of cytotoxicity following transfection into HeLa P4/R5 cells.

### Example 3

### NEIL3 siRNAs Inhibit Production of Infectious Virus

In the original screen, the siRNAs were transfected into the same cells that were used to assess viral infectivity. As a result, any siRNAs that directly affected transcription, translation, or enzymatic activity of the LTR-driven β-galactosidase reporter genes would affect the outcome of the assay and be recorded as hits. To eliminate these hits, an assay was developed in which HeLa P4/R5 cells were transfected with siRNAs, infected with virus, and then virus that was shed after several rounds of infection was used to infect freshly plated HeLa (P4/R5) cells.

Day 1: HeLa (P4/R5) cells were plated at 1000 cells per well into 384-well Falcon plates (Cat# 353988).

Day 2: HeLa (P4/R5) cells were transfected with siRNA pools as follows:
1. siRNAs, including the siRNA pool for NEIL3 described in Example 1 were transfected in triplicate at a final concentration of 50 nM using Oligofectamine (Invitrogen) at a final concentration of 0.5%. The same positive and negative control siRNAs were used as well. One column of mock-transfected cells with no virus added and one column with no cells plated were included on each plate as controls representing the bottom of the assay (lowest potential signal).
2. Oligofectamine was mixed with Optimem in a 1:50 ratio
3. 20 μL of Optimem:Oligofectamine mixture was dispensed into each well of a sterile 384-well plate.
4. 1 μL of siRNA (10 μM) was transferred from each well of the siRNA stock plate into the Optimem/Oligofectamine-containing plates and the resultant solution was mixed up and down five times.
5. The plate was incubated at room temperature for 10 minutes.
6. 5 μL of the siRNA-oligofectamine complex was added to each well of the HeLa(P4/R5) cells

Day 3: Transfected HeLa(P4/R5) cells were infected with HXB2 HIV in the presence of an integrase inhibitor as follows:
1. Media was removed from the cells for two out of the three transfection plates;
2. 40 μL fresh media was added to each well;
3. The integrase inhibitor was diluted to 20 nM in media. 20 μL of the 20 nM solution was added to each well of two of the plates (the final concentration of the integrase inhibitor was equal to the IC50 of the compound for inhibition of viral infection in this assay (Anthony et al, 2004). 20 μL of media without compound was added to the remaining two plates;
4. HXB2 HIV was diluted with media 200×. 20 μL of diluted HXB2 was added to each well; and
5. Viral infection was allowed to proceed for 96 h.

Day 7: Virus was removed from the cells after multiple infection cycles and used to infect a second plate of HeLa (P4/R5) cells.
1. A second plate of HeLa (P4/R5) cells was plated out at 2000 cells/well into a 384 well dish.
2. 20 μL of media from the plate transfected with siRNA and infected with HXB2 HIV was transferred to the second plate of cells.

Day 9: Beta-galactosidase activity was measured as described in Example 1.

Readings for each plate were normalized to the reading for the luciferase negative control and expressed as “% of Luciferase Control”. Hits were considered to be those siRNA pools that inhibited β-galactosidase activity by at least 40% relative to luciferase. The experiment was carried out in triplicate and the NEIL3 siRNA pool was found to inhibit production of HIV by 63.6+/−10%. NEIL3 was then evaluated further with respect to tissue distribution to determine whether it could play an essential role in HIV infection in HeLa cells.

### Example 4

### NEIL3 Tissue Distribution

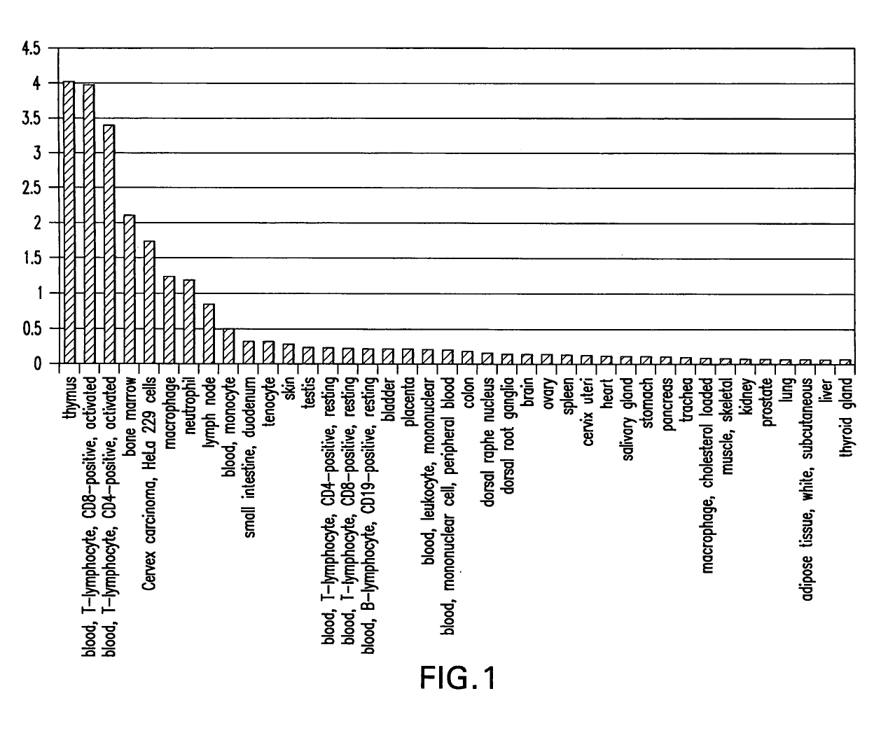

siRNAs chosen for further analysis were subsequently examined for expression in cells infected by HIV, or tissues that harbor the virus, including CD4+ T-lymphocytes, macrophage, lymph node and thymus using Merck's proprietary Body Atlas, which contains data from microarray experiments carried out with many different tissues compared against a species-specific reference pool. NEIL3 was found to be expressed almost exclusively in hematopoietic cells, including CD4+ T-cells and macrophages, as seen in FIG. 1.

NEIL3 siRNAs were then further evaluated in assays designed to determine the point in the viral life cycle at which the siRNAs interfered. These assays are described in the following examples.

### Example 5

### NEIL3 siRNAs Have No Effect on Viral Entry

Viral entry was assayed using a virus-like particle containing β-lactamase reporter protein (Tobiume et al., 2003).

Methods:

Day 1: HeLa P4/R5 cells were plated at 3000 cells/well in 80 μl media in 96-well plate.

Day 2: Cells were transfected with siRNAs as follows:
1. siRNAs from Dharmacon SMARTpools (used in Examples 5-7) were transfected at a final concentration of 50 nM using Oligofectamine (Invitrogen) at a final concentration of 0.5%.
2. 66 μL of Optimem/well was dispensed into a sterile 96-well plate, leaving the 12^{th} column empty.
3. 2 μL of siRNA (resuspended at 10 μM) was transferred from each well of the siRNA stock plate into the Optimem-containing plates such that the siRNA from well A3 of the mother plate was transferred into well A2 of the daughter plate, i.e., 2 μL of siRNA from each well was transferred into the corresponding plate into the same row position and the N-1 column position.
4. The resultant solution was mixed by pipetting up and down.
5. 240 μL Oligofectamine and 1210 μL Optimem was added to a tube, which was then incubated 5 minutes at room temperature.
6. 12 μL of the oligofectamine was dispensed to each well and mixed by pipetting up and down. The plate was incubated at room temperature for 15 minutes.
7. 20 μL of the siRNA-oligofectamine complex was added to each well of the HeLa (P4/R5) cells.

Day 3:
1. At 24 hours after transfection, the medium was removed and replaced with medium containing HIV recombinant particles (HIV virus-like particles containing a β-lactamase reporter construct as in Tobiume et al., 2003) at 100 μL per well. The infection was incubated at 37° C. for 3 hours.
2. The virus was removed by aspiration after 3 h and 100 p. 1 DMEM media (with no phenol red) was added.
3. 20 μL CCF4-AM mixtures from GeneBlazer (Invitrogen) was then added to each well with the final concentration of CCF4-AM at 5 μM. The plate was sealed and developed overnight at room temperature in the dark.

Day 4: β-lactamase activity within the cell is an indication of viral entry and delivery of viral contents internal to the capsid. Its activity was determined by measuring conversion of the CCF4-AM dye by reading fluorescence with excitation at 405 nm and emission at 460 nm (blue, cleaved CCF4-AM) or 535 nm (green, uncleaved CCF4-AM).

Transfection with NEIL3 siRNAs was not shown to affect fusion of β-lactamase-containing virus-like particles. The positive control siRNA pools, which targeted CD4 and CXCR4, inhibited particle fusion by at least 50% in multiple experiments.

### Example 6

### NEIL3 Has No Effect on Tat-Mediated LTR Transactivation

The effect of NEIL3 siRNAs on tat-mediated LTR transactivation was assayed by testing the effect of the siRNAs on expression of the LTR-β-galactosidase reporter gene in HeLa P4/R5 cells following transfection of a tat expression vector. The experiment was carried out as follows:

Day 1: HeLa (P4/R5) cells were plated at 3000 cells per well into 96-well Falcon plates;

Day 2: Cells were transfected with siRNAs as follows:
1. siRNAs were transfected at a final concentration of 100 nM using Oligofectamine (Invitrogen) at a final concentration of 0.5%. Positive and negative control siRNAs as used in Example 1 were utilized. NEIL3 siRNAs were obtained from Dharmacon SMARTpools, as indicated above.
2. 66 μL of Optimem per well was dispensed into a sterile 96-well plate, leaving the 12^{th} column empty.
3. 2 μL of siRNA (resuspended at 10 μM) was transferred from each well of the siRNA stock plate into the Optimem-containing plates such that the siRNA from well A3 of the mother plate was transferred into well A2 of the daughter plate (2 μL of siRNA from each well was transferred into the corresponding plate into the same row position and the N-1 column position).
4. The resultant solution was mixed by pipetting up and down.
5. 240 μL Oligofectamine was combined with 1210 μL Optimem in a tube, which incubated 5 minutes at room temperature.
6. 12 μL of the oligofectamine was dispensed into each well and mixed by pipetting up and down. The plate was incubated at room temperature for 15 minutes.
7. 20 μL of the siRNA-oligofectamine complex was added to each well of the HeLa (P4/R5) cells.

Day 4: The transfected HeLa(P4/R5) cells were transfected with pUCd5-Tat, an HIV1-tat expression vector. The transfection mixture was prepared in bulk for the entire plate as follows:
1. Mix 1 was prepared (one plate, 100 samples)

| | |
|---|---|
| a. Lipofectamine 2000 | 50 μL |
| b. Opti-MEM | 2500 μL |

2. Incubated at room temperature for 5 minutes.
3. Mix 2 was prepared (one plate, 100 samples)

| | |
|---|---|
| a. Opti-MEM | 2500 μL |
| b. PUC-D5 Tat (1 ng/μL) | 10 μL |

4. Incubated at RT for 5 minutes.
5. Mix 3 was prepared by combining Mix 1 and Mix 2, and the resultant mixture was incubated at room temperature for 20 minutes.
6. Transfection: Removed media from wells, add 50 μL of Mix 3 and 50 μL of culture media to each well, incubated for 24 hrs.

Day 5: Beta-galactosidase activity was measured as follows:
1. Media was removed from the cells.
2. Cells were washed with 40 μL PBS per well.
3. 20 μL of PBS was added to each well.
4. 20 μL of Lysis buffer plus substrate (25:1) (Applied Biosystems, Cat# GSY10,000) were then added to each well and the plates were incubated at room temperature in the dark for 30 minutes.
5. The plates were read using a VictorLight luminometer (PerkinElmer)

NEIL3 siRNAs were assayed in triplicate and found to reduce Tat-mediated transcription by 35%. Due to the fact that the effects on HIV replication were more substantial (60-70%), the effects on Tat-based transcription were considered unlikely to be the primary role of NEIL3 in HIV replication.

### Example 7

### Effect of NEIL3 on Reverse Transcription and Integration of Viral DNA

The effect of NEIL3 siRNAs on reverse transcription and integration of viral DNA (vDNA) was assessed using Taqman quantification of full length vDNA and integrated vDNA as described in Butler et al (2001), with modifications to the assay to allow for siRNA transfection:

Day 1: HeLa (P4/R5) cells were seeded at 128,000 cells per well in a 6-well plate containing 1.6 mL media.

Day 2: Cells were transfected with siRNAs as follows:
1. siRNAs were transfected at a final concentration of 100 nM using Oligofectamine (Invitrogen) at a final concentration of 0.5%. Positive and negative control siRNAs were CD4 and luciferase, respectively. NEIL3 siRNAs were obtained from Dharmacon.
2. 804 μL of Optimem was dispensed into a sterile microfuge tube.
3. 12 μL of siRNA (resuspended at 20 μM) was transferred into the Optimem-containing microfuge tubes.
4. The resultant solution was mixed by pipetting up and down.
5. 600 μL Oligofectamine and 3000 μL Optimem were combined in a second tube, which incubated 5 minutes at room temperature.
6. 144 μL of the oligofectamine mixture was added to each siRNA mixture and mixed by pipetting up and down followed by an incubation step at room temperature for 15 minutes.
7. Added 4004 of the siRNA-oligofectamine complex to each well of the HeLa(P4/R5) cells. Incubated overnight at 37 C, 5% CO2.

Day 3: Infected cells with HIV:
1. Media was removed from each well and fresh media was added.
2. 1 mL of HXB2 HIV diluted 1:50 was added to each well.

Day 5: DNA was extracted using the DNEasy Blood and Tissue Kit according to the manufacturer's instructions (Qiagen, cat# 69504).
1. Reverse transcription was quantified using the following parameters:

| | | |
|---|---|---|
| 2x TaqMan universal mix | | 10 μL |
| 10 μM 5NCR forward primer | | 1 μL |
| 5′GGCTAACTAGGGAACCCACTGCTT-3′ | (SEQ ID NO: 11) | |
| 10 μM 5 NCR reverse primer | | 1 μL |
| 5′-AGCCGAGTCCTGCGTCG-3′ | (SEQ ID NO: 12) | |
| 10 μM 5 NCR probe (FAM/TAMRA) | | 1 μL |
| 5′-(FAM)-CCTCAATAAAGCTTGCCTTGAG | (SEQ ID NO: 13) | |
| TGCTTCAA-(TAMRA)-3′ | | |
| water | | 2 μL |
| sample DNA | | 5 μL |
| Reaction cycles: | | |
| 50° C. for 2 min. | | |
| 95° C. for 5 min | | |
| 45 cycles of | | |
| 95° C. for 15 sec | | |
| 60° C. for 1 min | | |

2. Integration was quantified using the parameters outlined in Butler et al. (2(

| | | |
|---|---|---|
| 2x TaqMan universal mix | | 10 μL |
| 10 μM Alu/LTR forward primer | | 1 μL |
| 5′-AACTAGGGAACCCACTGCTTAAG-3′ | (SEQ ID NO: 14) | |
| 10 μM Alu/LTR reverse primer | | 1 μL |
| 5′-TGCTGGGATTACAGGCGTGAG-3′ | (SEQ ID NO: 15) | |
| 10 μM Alu/LTR | | 1 μL |
| 5′-(FAM)-ACACTACTTGAAGCACTCAA | (SEQ ID NO: 16) | |
| GGCAAGCTTT-(TAMRA)-3′ | | |
| water | | 2 μL |
| sample DNA | | 5 μL |
| Reaction cycles: | | |
| 50° C. for 2 min | | |
| 95° C. for 10 min | | |
| 45 cycles of: | | |
| 95° C. for 15 sec | | |
| 60° C. for 1 min 30 sec | | |

3. Cytotoxicity was quantified using the following parameters:

| | | |
|---|---|---|
| 2X Taqman universal mix | | 10 μL |
| 10 μM Cytox forward primer | | 1 μL |
| 5′-TCCGCTACCATAATCATCGCT-3′ | (SEQ ID NO: 17) | |
| 10 μM Cytox reverse primer | | 1 μL |
| 5′-CCGTGGAGTGTGGCGAGT-3′ | (SEQ ID NO: 18) | |
| 10 μM Cytox probe | | 1 μL |
| 5′-(VIC)-TCCCCACCGGCGTCAAAGTA | (SEQ ID NO: 19) | |
| TTTAGC-(TAMRA)-3′ | | |
| water | | 2 μL |
| sample DNA | | 5 μL |

All data were normalized for cell number/cytotoxicity using the cytotoxicity results. Inhibition was determined by comparing normalized data from NEIL3 siRNA transfected cells to controls. NEIL3-targeting siRNAs reduced the production of viral DNA to 40% of background levels (+/−6%, N=2), which is close to the overall level of inhibition of HIV infection observed after NEIL3 knockdown. Thus, NEIL3 appears to function at the level of reverse transcription or vDNA production. Additionally, integrated viral DNA levels were reduced to 16.5% of background levels (+/−8%, N=2), which suggests NEIL3 may also play a role in nuclear localization or integration of viral DNA.

Validation of the role of NEIL3 in HIV infection was carried out by rescuing the siRNA-triggered knockdown of HIV infectivity through expression of a NEIL3 cDNA that was not affected by the siRNAs used to knock down the endogenous NEIL3 mRNA. This experiment was carried out as described in Example 8 below:

### Example 8

### Rescue of NEIL3 siRNA-Mediated Inhibition of HIV Replication with Expression of a Non-Targeted NEIL3 cDNA

The following procedures were carried out:
Day 1: HeLa (P4/R5) cells were plated at 3000 cells per well into 96-well Falcon plates
Day 2: HeLa (P4/R5) cells were co-transfected with siRNA against the 3′UTR and cDNA of the tested gene as follows:

1. siRNAs targeting the 3′UTR of NEIL3 were co-transfected with cDNA expressing NEIL3 that did not include the 3′UTR. cDNA3.1 N-V5 DEST vector (Invitrogen) was included as negative control for the same amount of NEIL3 in the same vector. The sequence included in the cloneNEIL3 ORF clone was obtained from Invitrogen (Ultimate™ ORF Clone; IOH3978). Transfections were carried out in triplicate with Lipofectamine 2000 (Invitrogen) at a final concentration of 1%. siRNA final concentration is 50 nM, plasmid DNA is 100 ng/well.

NEIL3 3′UTR siRNAs were designed using the publicly available tool provided by Dharmacon at: http://www.dharmacon.com/sidesign/defaultaspx:

| | |
|---|---|
| siRNA #1: UGAUGAACGUUCUAUGUAUdTdT | (SEQ ID NO: 20) |
| siRNA #2: CUAUGUAUUUCAUCGGAUAdTdT | (SEQ ID NO: 21) |
| siRNA #3: GGAUUAUGCGACACAAUAAdTdT | (SEQ ID NO: 22) |

For each transfection:
2. 1 μL Lipofectamine 2000 and 24 μL of Opti-MEM were mixed at room temperature for 5 minutes.
2. 23.5 μL of Opti-MEM was mixed with 0.5 μL of 10 uM siRNA and 1 μL of 100 ng/μl cDNA at room temperature for 5 minutes.
4. The mixture from step 2 and step 3 was combined and incubated at room temperature for 20 minutes.
5. 50 μL of the complex from step 4 was transferred to cells with 50 μL of culture media (DMEM+10% serum) to a final volume 100 μL and incubated at 37° C. for 6 hours.
7. The media was then removed with transfection complex, and changed to fresh culture media, which incubated at 37° C. overnight.

Day 3: Transfected HeLa(P4/R5) cells were infected with HXB2 HIV as follows:
1. Media was removed from the cells;
2. HXB2 HIV was diluted with media 400×. 100 μL of diluted HXB2 was added to each well; and
3. Viral infection was allowed to proceed for 48 h.

Day 5: Beta-galactosidase activity was measured as follows:
1. 100 μL of Lysis buffer plus substrate (25:1)(Applied Biosystems, Cat# GSY10,000) was added to each well and the plates were incubated at room temperature in the dark for 30 minutes.
2. The plates were read using a VictorLight luminometer (PerkinElmer).

Three independent siRNAs targeting the 3′UTR of NEIL3 were seen to inhibit HIV infection to between 11 and 22% of levels after transfection of a nonsilencing siRNA. Co-transfection of these siRNAs with an expression vector for NEIL3 cDNA lacking the 3′UTR (so the expressed cDNA can not be silenced by 3′UTR-targeting siRNAs) resulted in recovery of HIV infectivity in the cells. The degree of recovery ranged from 89-96% infectivity relative to controls in which cells were transfected with a nonsilencing siRNA.

### Example 9

### MUTYH siRNAs Inhibit HIV Infection

MUTYH was identified as being associated with HIV infection in an siRNA screen in which DNA repair factors were specifically targeted to determine their role in HW infection. Like NEIL3, MUTYH is a DNA glycosylase associated with the base excision repair pathway, further implicating this pathway in HIV infection. The following experiments were carried out to confirm the original observation that transfection of siRNAs targeting MUTYH decreases HIV infection.

Day 1: HeLa (P4/R5) cells were plated at 2000 cells per well in 4×96-well plates.

Day 2: HeLa (P4/R5) cells were transfected with siRNA pools as follows:
1) siRNAs were transfected at a final concentration of 100 nM using Oligofectamine (Invitrogen) at a final concentration of 0.5%. Positive and negative control siRNAs are included as follows:
   CDK9 (positive control): GUGGUCAACUUGAUUGAGAdTdT (SEQ ID NO: 23)
   Cyclin T1 (positive control): (purchased from Santa Cruz Biotechnology (cat #sc-35144) and luciferase, used as the negative control, as in Example 1.

| MUTYH siRNA #1: | |
|---|---|
| GUGAUGGGAUGAUUGCUGATT | (SEQ ID NO: 24) |
| MUTYH siRNA #2: | |
| GCUGACAUAUCAAGUAUAUTT | (SEQ ID NO: 25) |
| MUTYH siRNA #3: | |
| CUCAUACCAUCUAUUCAGATT | (SEQ ID NO: 26) |
| MUTYH siRNA #4: | |
| CUCACAUCAAGCUGACAUATT | (SEQ ID NO: 27) |
| MUTYH siRNA #5: | |
| CACACCUUCUCUCACAUCATT | (SEQ ID NO: 28) |
| MUTYH siRNA #6: | |
| GCUGUUUCCACCGCCAUGATT | (SEQ ID NO: 29) |

3. 66 μL of Optimem/well was dispensed into a sterile 96-well plate, leaving the 12^{th} column empty.
4. 2 μL of siRNA (resuspended at 10 μM) was transferred from each well of the siRNA stock plate into the Optimem-containing plates, such that the siRNA from well A3 of the mother plate is transferred into well A2 of the daughter plate (2 μL of siRNA from each well is transferred into the corresponding plate into the same row position and the N-1 column position).
5. The resultant solution was mixed by pipetting up and down.
6. 240 μL Oligofectamine and 1210 μL Optimem were then added to a microfuge tube, which was incubated 5 minutes at room temperature.
7. 12 μL of the oligofectamine was dispensed to each well and mixed by pipetting up and down. The plate was then incubated at room temperature for 15 minutes.

8. 20 μL of the siRNA-oligofectamine complex was added to each well of the HeLa(P4/R5) cells.

Day 3: Transfected HeLa(P4/R5) cells were infected with HXB2 HIV in the presence and absence of an integrase inhibitor as follows:
1. Media was removed from the cells
2. 80 μL fresh media was added to each well
3. An integrase inhibitor was diluted to 20 nM in media. 40 μL of the 20 nM integrase inhibitor solution was added to each well of two of the plates (the final concentration of the integrase inhibitor was equal to the IC50 of the compound for inhibition of viral infection in this assay (Anthony et al, 2004). Forty μL of media without compound was added to the remaining two plates.
4. HXB2 HIV was diluted with media 100× and forty μL of diluted HXB2 was added to each well.
5. Viral infection was allowed to proceed for 48 h.

Day 5. Beta-galactosidase activity was measured as follows:
1. Media was removed from the cells.
2. Cells were washed with 200 μL PBS per well.
3. 20 μL Lysis Buffer (Galacto-Light Plus, Tropix, cat# BL100 P) containing DTT was added to each well, and the plates were shaken for 10 minutes.
4. 80 μL of substrate was then added to each well and the plates were incubated at room temperature in the dark for 1 hour.
5. 100 μL of enhancing solution was added to each well and the plates were read using a Dynex luminometer.

Readings for each plate were normalized to the reading for the luciferase negative control and expressed as “% of Luciferase Control”. Hits were considered to be those siRNA pools that suppressed beta-galactosidase activity by 40% or more, or those that showed 30% or greater inhibition of beta-galactosidase activity in the presence of IC50 levels of the integrase inhibitor, compared to the activity in the absence of compound treatment.

Three out of the six siRNAs tested (siRNA #s 2, 4, and 5) were found to decrease HIV infectivity by more than 50%.

### Example 10

### MUTYH siRNAs are not Cytotoxic

Because MUTYH siRNAs were identified via their inhibition of HIV infectivity, it was important to rule out that these siRNAs appeared to hit in the infectivity screen simply due to cytotoxicity. For this reason, the MUTYH siRNA pool was examined for cytotoxic effects in the cytotoxicity assay described in Example 1. siRNAs that led to viability levels of less than 70% as determined by Alamar Blue fluorescence were considered to be cytotoxic. It was found that the MUTYH siRNAs did not show any evidence of cytotoxicity following transfection into HeLa P4/R5 cells.

### Example 11

### MUTYH siRNAs are Effective in Decreasing MUTYH mRNA Levels

To demonstrate the effectiveness of the MUTYH-targeting siRNAs, the siRNAs were transfected into cells and the cellular mRNA levels of MUTYH were assessed by RT-PCR as described below:

Day 1: HeLa (P4/R5) cells were plated at 3000 cells per well into 96-well Falcon plates.

Day 2: Cells were transfected with siRNAs as follows:
1. siRNAs were transfected at a final concentration of 100 nM using Oligofectamine (Invitrogen) at a final concentration of 0.5%.
   MUTYH siRNAs tested:

| MUTYH siRNA #7: | |
|---|---|
| UGAGAAUCCUGUUGUUAGUdTdT | (SEQ ID NO: 30) |
| MUTYH siRNA #8: | |
| CCUGAGAAUCCUGUUGUUAdTdT | (SEQ ID NO: 31) |
| MUTYH siRNA #9: | |
| GAGAAUCCUGUUGUUAGUAdTdT | (SEQ ID NO: 32) |

MUTYH siRNA pool: an equimolar combination of MUTYH siRNAs #1,
   2 and 3 from Example 9 above.
3. 66 μL of Optimem/well was dispensed into a sterile 96-well plate, leaving the 12^{th} column empty.
4. 2 μL of siRNA (resuspended at 10 μM) was transferred from each well of the siRNA stock plate into the Optimem-containing plates, such that the siRNA from well A3 of the mother plate is transferred into well A2 of the daughter plate (2 μL of siRNA from each well is transferred into the corresponding plate into the same row position and the N-1 column position).
5. The resultant solution was mixed by pipetting up and down.
6. 240 μL Oligofectamine and 12104 Optimem were added to a microfuge tube and incubated 5 minutes at room temperature.
7. 12 μL of the oligofectamine was dispensed to each well and mixed by pipetting up and down. The plate was incubated at room temperature for 15 minutes.
8. 20 μL of the siRNA-oligofectamine complex was added to each well of the plate containing the HeLa (P4/R5) cells.

Day 5: RNA was harvested using the RNeasy kit (Qiagen, cat# 74181), according to the manufacturer's instructions

MUTYH mRNA levels were quantified by RT-PCR in a reaction including:

| | |
|---|---|
| Cellular RNA sample | 10 μL |
| EZ RT-PCR Core Reagents kit (ABI, cat# N8080236) | 15 μL |
| MUTYH Primer/probe set (ABI, cat# Hs00276113) | 1.25 μL |
| 50° C. for 2 min. | |
| 60° C. for 30 min. | |
| 95° C. for 5 min. | |
| 40 cycles of 94° C. for 20 sec. and 62° C. for 1 min. | |

As an internal control, cyclophilin mRNA levels were measured in the same samples using the following reaction:

| | |
|---|---|
| Cellular RNA sample | 10 μL |
| EZ RT-PCR Core Reagents kit (ABI, cat# N8080236) | 15 μL |
| PPIA Primer/probe set (ABI, cat# 4326316E) | 1.25 μL |
| 50° C. for 2 min. | |
| 60° C. for 30 min. | |
| 95° C. for 5 min. | |
| 40 cycles of 94° C. for 20 sec. and 62° C. for 1 min. | |

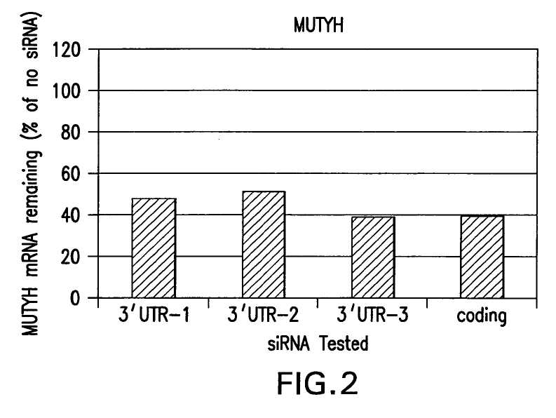

MUTYH mRNA levels were normalized to cyclophilin levels to control for cell number. The normalized MUTYH level after siRNA transfection was compared with untransfected cells to determine the percent mRNA knockdown. MUTYH-targeting siRNAs were shown to knock down MUTYH mRNA levels to less than 50% of wild type levels, as indicated in FIG. 2.

### Example 12

### MUTYH Tissue Distribution

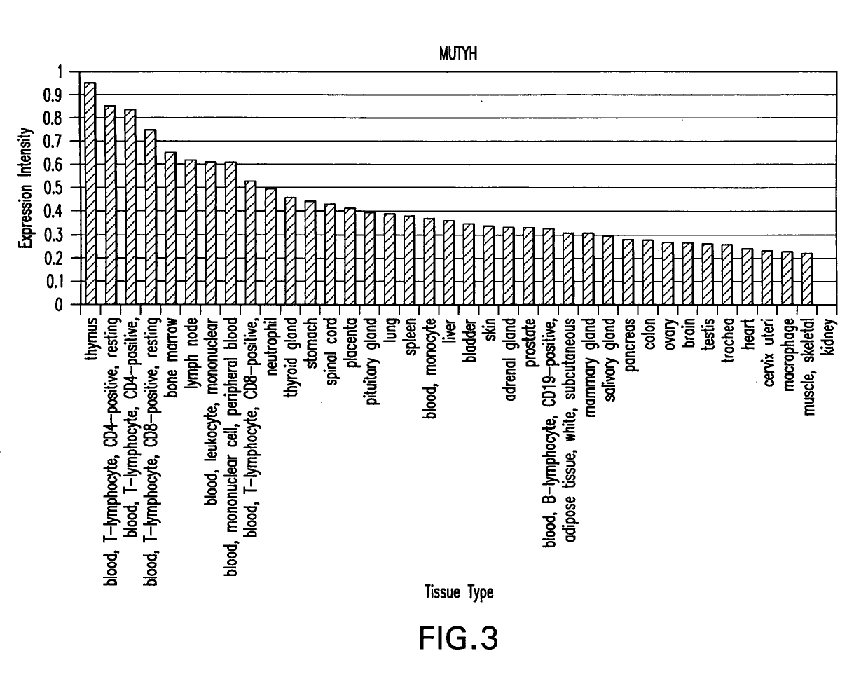

siRNAs chosen for further analysis were examined for expression in cells infected by HIV or tissues that harbor the virus, including CD4+ T-lymphocytes, macrophage, lymph node and thymus using Merck's proprietary Body Atlas, which contains data from microarray experiments carried out with many different tissues compared against a species-specific reference pool. MUTYH was found to be expressed in most tissues with the exception of kidney; highest expression is in thymus and T-cells, as seen in FIG. 3.

### Example 13

### Induction of MUTYH mRNA Levels in HeLa P4/R5 Cells After HIV Infection

A protein that is required for viral infection in a cell may be induced to higher expression levels upon infection with that virus. For this reason, MUTYH mRNA levels were assessed, using the RT-PCR procedure described in Example 11, after HeLa P4/R5 cell infection with HXB2 HIV for 0, 2, 4, 24, and 48 hours post infection.

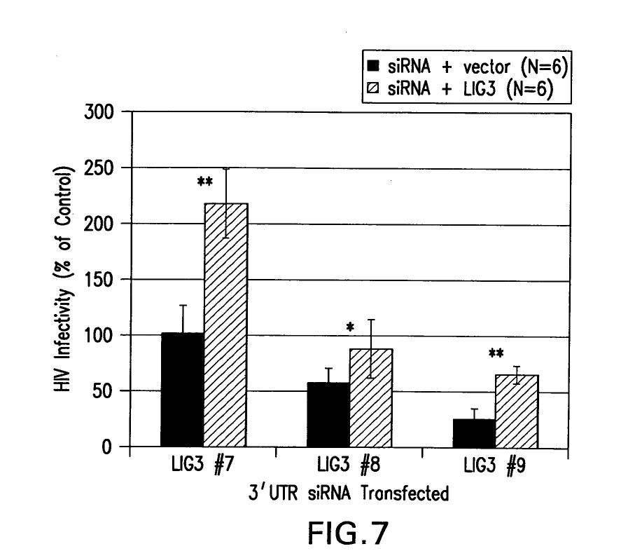

It was determined that MUTYH has a low level, transient increase in expression beginning 4 hours post-HIV infection, as seen in FIG. 7. MUTYH siRNAs were subsequently evaluated in assays designed to determine the point in the viral life cycle at which the siRNAs interfered, as described below.

### Example 14

### Effect of MUTYH on Tat-Mediated LTR Transactivation

The effect of MUTYH siRNAs on tat-mediated LTR transactivation was assayed by testing the effect of the siRNAs on expression of the LTR-β-galactosidase reporter gene in HeLa P4/R5 cells following transfection of a tat expression vector. The experiment was carried out as follows:

Day 1: HeLa (P4/R5) cells were plated at 3000 cells per well into 96-well Falcon plates

Day 2: Cells were transfected with siRNAs as follows:

1. siRNAs were transfected at a final concentration of 100 nM using Oligofectamine (Invitrogen) at a final concentration of 0.5%. Positive and negative control siRNAs and MUTYH siRNA were the same as in Example 9. The MUTYH siRNA was a SMARTpool from Dharmacon.

2. 66 μL of Optimem/well was dispensed into a sterile 96-well plate, leaving the 12^{th} column empty.

3. 2 μL of siRNA (resuspended at 10 μM) from each well of the siRNA stock plate was transferred into the Optimem-containing plates such that the siRNA from well A3 of the mother plate is transferred into well A2 of the daughter plate (2 μL of siRNA from each well is transferred into the corresponding plate into the same row position and the N-1 column position).

4. The resultant solution was mixed by pipetting up and down.

5. 240 μL Oligofectamine and 1210 μL Optimem were added to a microfuge tube which incubated 5 minutes at room temperature.

6. 12 μL of the resultant mix was dispensed to each well and mixed by pipetting up and down. The plate was incubated at room temperature for 15 minutes.

7. 20 μL of the siRNA-oligofectamine complex was added to each well of the plate containing the HeLa (P4/R5) cells.

Day 4: Transfected HeLa(P4/R5) cells were transfected with pUCd5-Tat, and HIV 1-tat expression vector. The transfection mixture was prepared in bulk for the entire plate as follows:
1. Mix 1 was prepared (one plate, 100 samples), which consisted of:

| | |
|---|---|
| a. Lipofectamine 2000 | 50 μL |
| b. Opti-MEM | 2500 μL, |

which was incubated at room temperature for 5 minutes.
2. Mix 2 was prepared (one plate, 100 samples), which consisted of:

| | |
|---|---|
| c. Opti-MEM | 2500 μL |
| d. PUC-D5 Tat (1 ng/μL) | 10 μL, |

which was incubated at room temperature for 5 minutes.
3. Mix 3 was prepared, which combined Mix 1 and Mix 2 in their entirety, and was then incubated at room temperature for 20 minutes.
4. For the transfection step, media was removed from the wells, 50 pt of Mix 3 and 50 μL of culture media was added to each well and incubated for 24 hrs.

Day 5: Beta-galactosidase activity (an indication of viral infection) was measured as follows:
1. Media was removed from the cells.
2. Cells were washed with 40 μL PBS per well.
3. 20 μL of PBS was added to each well.
4. 20 μL of Lysis buffer plus substrate (25:1) (Applied Biosystems, Cat# GSY10,000) were then added to each well and the plates were incubated at room temperature in the dark for 30 minutes.
5. The plates were read using a VictorLight luminometer (PerkinElmer)
MUTYH siRNAs were assayed in triplicate and found to reduce Tat-mediated transcription by 54%.

### Example 15

### Effect of MUTYH on Reverse Transcription and Integration of Viral DNA

The effect of MUTYH siRNAs on reverse transcription and integration of viral DNA was assessed using Taqman quantification of full length vDNA and integrated vDNA as described in Butler et al (2001) with modifications to the assay to allow for siRNA transfection:

Day 1: HeLa (P4/R5) cells were seeded at 128,000 cells per well in a 6-well plate containing 1.6 mL media.

Day 2: Cells were transfected with siRNAs as follows:

1. siRNAs were transfected at a final concentration of 100 nM using Oligofectamine (Invitrogen) at a final concentration of 0.5%. Positive and negative control siRNAs were CD4 and luciferase, respectively; the MUTYH siRNAs were obtained from Dharmacon.

2. 804 μL of Optimem was dispensed into a sterile microfuge tube.

3. 12 μL of siRNA (resuspended at 20 μM) was transferred into the Optimem-containing microfuge tubes and mixed by pipetting up and down.

4. In a second tube, 600 μL Oligofectamine was combined with 3000 μL Optimem and incubated 5 minutes at room temperature.

5. 144 μL of the oligofectamine mixture was dispensed to each siRNA mixture and mixed by pipetting up and down. The tube was then incubated at room temperature for 15 minutes.

6. 400 μL of the siRNA-oligofectamine complex was then added to each well of plate containing the HeLa(P4/R5) cells and it was incubated overnight at 37 C, 5% CO₂.

Day 3: Infected cells with HIV:

1. Media was removed from each well and fresh media was added.

2. 1 mL of HXB2 HIV diluted 1:50 was added to each well.

Day 5: DNA was extracted using DNEasy Blood and Tissue Kit according to the manufacturer's instructions (Qiagen, cat# 69504).

Reverse transcription was quantified using the following parameters:

| | | |
|---|---|---|
| 2x TaqMan universal mix | | 10 μL |
| 10 μM 5NCR forward primer | | 1 μL |
| 5′GGCTAACTAGGGAACCCACTGCTT-3′ | (SEQ ID NO: 11) | |
| 10 μM 5 NCR reverse primer | | 1 μL |
| 5′-AGCCGAGTCCTGCGTCG-3′ | (SEQ ID NO: 12) | |
| 10 μM 5 NCR probe (FAM/TAMRA) | | 1 μL |
| 5′-(FAM)-CCTCAATAAAGCTTGCCTT | (SEQ ID NO: 13) | |
| GAGTGCTTCAA-(TAMRA)-3′ | | |
| water | | 2 μL |
| sample DNA | | 5 μL |
| Reaction cycles: | | |
| 50° C. for 2 min. | | |
| 95° C. for 5 min | | |
| 45 cycles of | | |
| 95° C. for 15 sec | | |
| 60° C. for 1 min | | |

Integration was quantified using the parameters outlined in Butler et al. (2001

| | | |
|---|---|---|
| 2x TaqMan universal mix | | 10 μL |
| 10 μM Alu/LTR forward primer | | 1 μL |
| 5′-AACTAGGGAACCCACTGCTTAAG-3′ | (SEQ ID NO; 14) | |
| 10 μM Alu/LTR reverse primer | | 1 μL |
| 5′-TGCTGGGATTACAGGCGTGAG-3′ | (SEQ 1D NO: 15) | |
| 10 μM Alu/LTR | | 1 μL |
| 5′-(FAM)-ACACTACTTGAAGCACTCA | (SEQ ID NO: 16) | |
| AGGCAAGCTTT-(TAMRA) 3′ | | |
| water | | 2 μL |
| sample DNA | | 5 μL |
| Reaction cycles: | | |
| 50° C. for 2 min | | |
| 95° C. for 10 min | | |
| 45 cycles of: | | |
| 95° C. for 15 sec | | |
| 60° C. for 1 min 30 sec | | |

Cytotoxicity was quantified using the following parameters:

| | | |
|---|---|---|
| 2X Taqman universal mix | | 10 μL |
| 10 μM Cytox forward primer | | 1 μL |
| 5′-TCCGCTACCATAATCATCGCT-3′ | (SEQ ID NO: 17) | |
| 10 μM Cytox reverse primer | | 1 μL |
| 5′-CCGTGGAGTGTGGCGAGT-3′ | (SEQ ID NO: 18) | |
| 10 μM Cytox probe | | 1 μL |
| 5′-(VIC)-TCCCCACCGGCGTCAAAGT | (SEQ ID NO: 19) | |
| ATTTAGC-(TAMRA)-3′ | | |
| water | | 2 μL |
| sample DNA | | 5 μL |
| Reaction cycles: | | |
| 95° C. for 10 min | | |
| Followed by 40 cycles of: | | |
| 95° C. for 16 sec | | |
| 55° C. for 30 sec | | |
| 60° C. for 30 sec | | |

All data were normalized for cell number/cytotoxicity using the cytotoxicity Taqman results. Inhibition was determined by comparing normalized data from MUTYH siRNA transfected cells to controls.

It was found that MUTYH-targeting siRNAs reduced the production of viral DNA to 53% of background levels (+/−17%, N=2), which is close to the overall level of inhibition of HIV infection observed after MUTYH knockdown. Thus, MUTYH appears to function at the level of reverse transcription or vDNA production. Additionally, integrated viral DNA levels were reduced to 69.5% of background levels (+/−16%, N=2). Validation of the role of MUTYH in HIV infection was carried out by rescuing the siRNA-triggered knockdown of HIV infectivity through expression of a MUTYH cDNA that was unaffected by the siRNAs used to knock down the endogenous MUTYH mRNA, as described below.

### Example 16

### Rescue of MUTYH siRNA-Mediated Inhibition of HIV Replication with Expression of a Non-Targeted MUTYH cDNA

Day 1: HeLa (P4/R5) cells were plated at 3000 cells per well into 96-well Falcon plates

Day 2: HeLa (P4/R5) cells were co-transfected with siRNA against the 3′UTR and cDNA of the tested gene as follows:
1. siRNAs targeting the 3′UTR of MUTYH were co-transfected with cDNA expressing MUTYH that did not include the 3′UTR. cDNA3.1 N-V5 DEST vector (Invitrogen) was included as a negative control for the same amount of MUTYH in the same vector. The ultimate MUTYH ORF clone was obtained from Invitrogen (IOH5124). Transfections were carried out in triplicate with Lipofectamine 2000 (Invitrogen) at a final concentration of 1%. siRNA final concentration is 50 nM, plasmid DNA is 100 ng/well.

MUTYH 3′UTR siRNAs were:

| | |
|---|---|
| siRNA #7: UGAGAAUCCUGUUGUUAGUdTdT | (SEQ ID NO: 30) |
| siRNA #8: CCUGAGAAUCCUGUUGUUAdTdT | (SEQ ID NO: 31) |
| siRNA #9: GAGAAUCCUGUUGUUAGUAdTdT | (SEQ ID NO: 32) |

For each transfection, the following steps were carried out:
2. 1 μL Lipofectamine 2000 was mixed with 24 μL of Opti-MEM and incubated at room temperature for 5 minutes.
3. 23.5 μL of Opti-MEM was mixed with 0.5 μL of 10 uM siRNA and 1 μl of 100 ng/μl cDNA and incubated at room temperature for 5 minutes.
4. The mixture from step 2 and step 3 was combined and incubated at room temperature for 20 minutes.
5. 50 μL of the complex from step 4 was transferred to cells with 50 μL of culture media (DMEM+10% serum) to a final volume of 100 μL and incubated at 37° C. for 6 hours.
6. The media with transfection complex was removed and changed to fresh culture media and incubated at 37° C. overnight.

Day 3: The transfected HeLa(P4/R5) cells were infected with HXB2 HIV as follows:
1. Media was removed from the cells
2. HXB2 HIV was diluted with media 400×. 1004 of diluted HXB2 was added to each well.
3. Viral infection was allowed to proceed for 48 h.

Day 5: Beta-galactosidase activity was measured as follows:
1. 100 μL of Lysis buffer plus substrate (25:1) (Applied Biosystems, Cat# GSY10,000) was added to each well and the plates were incubated at room temperature in the dark for 30 minutes.
2. The plates were read using a VictorLight luminometer (PerkinElmer).

Three independent siRNAs targeting the 3′UTR of MUTYH inhibited HIV infection to between 11 and 22% of levels after transfection of a nonsilencing siRNA. Co-transfection of these siRNAs with an expression vector for MUTYH cDNA lacking the 3′UTR (so the expressed cDNA can not be silenced by 3′UTR-targeting siRNAs) resulted in recovery of HIV infectivity in the cells. The degree of recovery ranged from 89-96% infectivity relative to controls in which cells were transfected with a nonsilencing siRNA.

### Example 17

### LIG3 siRNAs Inhibit HIV Infection

LIG3 is a DNA ligase associated with the base excision repair pathway. The following experiments were carried out to determine whether transfection of siRNAs targeting LIG3 decreases HIV infection.

The following procedures were carried out:
Day 1: HeLa (P4/R5) cells were plated at 2000 cells per well in 4×96-well plates.
Day 2: HeLa (P4/R5) cells were transfected with siRNA pools as follows:
1) siRNAs were transfected at a final concentration of 100 nM using Oligofectamine (Invitrogen) at a final concentration of 0.5%. Positive and negative control siRNAs are included as follows:
   CDK9 (positive control): GUGGUCAACUUGAUUGAGAdTdT (SEQ ID NO: 23)
   Cyclin T1 (positive control): (purchased from Santa Cruz Biotechnology (cat #sc-35144) and luciferase, which was used as a negative control as in Example 1.

| LIG3 siRNA #1: | |
|---|---|
| CGGAUCAUGUUCUCAGAAATT | (SEQ ID NO: 33) |
| LIG3 siRNA #2: | |
| GGAAGUGGAUGAGUUCCUUTT | (SEQ ID NO: 34) |
| LIG3 siRNA #3: | |
| CCAGGUGACUUCUCCAGUGTT | (SEQ ID NO: 35) |
| LIG3 siRNA #4: | |
| CAAUCAGAGUCUUCUUUGATT | (SEQ ID NO: 36) |
| LIG3 siRNA #5: | |
| CUCAUACAGCUGACGGGAUTT | (SEQ ID NO: 37) |
| LIG3 siRNA #6: | |
| GUUUACAACUUGAACGAUATT | (SEQ ID NO: 38) |

9. 66 μL of Optimem/well was dispensed into a sterile 96-well plate, leaving the 12^{th} column empty.

10. 2 μL of siRNA (resuspended at 10 μM) was transferred from each well of the siRNA stock plate into the Optimem-containing plates, such that the siRNA from well A3 of the mother plate is transferred into well A2 of the daughter plate, i.e., 2 μL of siRNA from each well is transferred into the corresponding plate into the same row position and the N-1 column position.

11. The resultant solution was mixed by pipetting up and down.

12. 240 μL Oligofectamine and 1210 μL Optimem were then added to a microfuge tube, which was incubated 5 minutes at room temperature.

13. 12 μL it of the oligofectamine was dispensed to each well and mixed by pipetting up and down. The plate was then incubated at room temperature for 15 minutes.

14. 20 μL of the siRNA-oligofectamine complex was added to each well of the HeLa(P4/R5) cells.

Day 3: Transfected HeLa(P4/R5) cells were infected with HXB2 HIV in the presence and absence of an integrase inhibitor as follows:
6. Media was removed from the cells
7. 80 μL fresh media was added to each well
8. An integrase inhibitor was diluted to 20 nM in media. 40 μL of the 20 nM integrase inhibitor solution was added to each well of two of the plates (the final concentration of the integrase inhibitor was equal to the IC50 of the compound for inhibition of viral infection in this assay (Anthony et al, 2004). Forty μL of media without compound was added to the remaining two plates.
9. HXB2 HIV was diluted with media 100× and forty μL of diluted HXB2 was added to each well.
10. Viral infection was allowed to proceed for 48 h.

Day 5. Beta-galactosidase activity was measured as follows:
6. Media was removed from the cells.
7. Cells were washed with 200 μL PBS per well.
8. 20 μL Lysis Buffer (Galacto-Light Plus, Tropix, cat# BL100P) containing DTT was added to each well, and the plates were shaken for 10 minutes.
9. 80 μL of substrate was then added to each well and the plates were incubated at room temperature in the dark for 1 hour.
10. 100 μL of enhancing solution was added to each well and the plates were read using a Dynex luminometer.

Readings for each plate were normalized to the reading for the luciferase negative control and expressed as “% of Luciferase Control”. Hits were considered to be those siRNA pools that suppressed beta-galactosidase activity by 40% or more, or those that showed 30% or greater inhibition of beta-galactosidase activity in the presence of IC50 levels of the integrase inhibitor compared to the absence of compound treatment. It was found that three out of the six siRNAs tested (siRNA #s 2, 5, and 6) were found to decrease HIV infectivity by more than 40%.

### Example 18

### LIG3 siRNAs are not Cytotoxic

The LIG3 siRNA pool was examined for cytotoxic effects in the cytotoxicity assay described in Example 1. siRNAs that led to viability levels of less than 70% as determined by Alamar Blue fluorescence were considered to be cytotoxic. LIG3 siRNAs did not show any evidence of cytotoxicity following transfection into HeLa P4/R5 cells.

### Example 19

### LIG3 siRNAs are Effective in Decreasing LIG3 Protein Levels

To demonstrate the effectiveness of the LIG3-targeting siRNAs, the siRNAs were transfected into cells and the cellular levels of LIG3 protein were assessed by Western blotting as described below:
Day 1: HeLa (P4/R5) cells were plated at 128,000 cells per well into 6-well Falcon plates
Day 2: Cells were transfected with siRNAs as follows:
1. siRNAs were transfected at a final concentration of 100 nM using Oligofectamine (Invitrogen) at a final concentration of 0.5%. LIG3 siRNAs tested were LIG3 siRNA #2; #5 and #6. Control siRNAs were the Cyclin T1 (positive control) and Luciferase (Negative control), as described above. 3354 of Optimem/well was dispensed into 5 microfuge tubes. 5 μL of siRNA (resuspended at 20 μM) was transferred into the Optimem-containing microfuge tubes.
2. The resultant solution was mixed by pipetting up and down.
3. 480 μL Oligofectamine and 2400 μL Optimem were added to a tube and incubated 5 minutes at room temperature.
4. 60 μL of the Oligofectamine/Optimem mixture was dispensed to each tube containing siRNA and mixed by pipetting up and down. The mixture was incubated at room temperature for 15 minutes.
5. 320 μL of the siRNA-oligofectamine complex was added to each well of the plate containing the HeLa (P4/R5) cells. Cells were incubated for 28 hours at 37° C.
   Day 3: Protein extracts were obtained by lysing the cells in REM buffer containing protease inhibitors. The protein extracts were run on a 10% Tris-Glycine gel, and transferred to a nitrocellulose membrane. The membrane was blocked with Li-Cor blocking buffer for 1 hr, then incubated overnight with anti-LIG3 (Gene Tex, MS-LIG33-PX1) diluted 1:1000, along with a beta-actin antibody (Sigma, Cat# A2066, included as an internal control) diluted 1:5000 in Li-Cor blocking buffer+0.1% Tween 20.
   Day 4. The membrane was washed with PBS+0.1% Tween 20. Labeled secondary antibodies for the primary anti-Lig3 (Donkey anti mouse 800 channel labeled, Rockland Inc. 610-732-124) and Beta-Actin (Donkey anti rabbit 700 channel labeled, Rockland Inc. 611-730-127) were both diluted 1:10000 in Li-Cor blocking buffer+0.1% Tween 20, and incubated with the membrane for 1 h at room temperature. The membrane was then washed with PBST, and the location and quantity of the detected proteins were determined by Li-Cor scan.

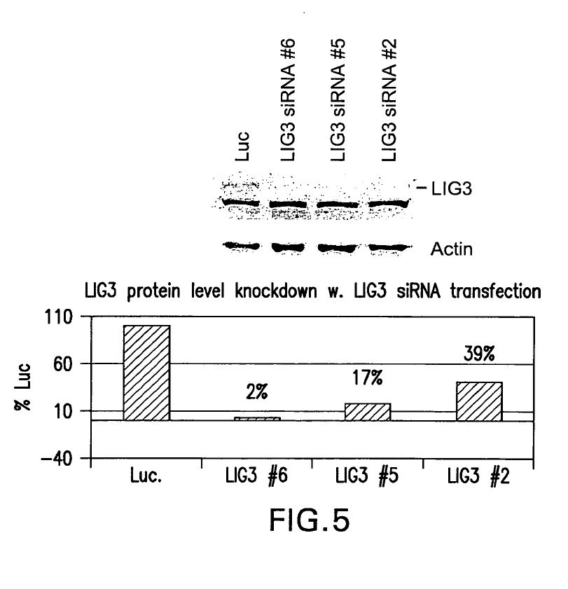

LIG3 protein levels were normalized to beta-actin levels to control for protein loading and transfer. The normalized LIG3 level after siRNA transfection was compared with untransfected cells to determine the percent knockdown. It was found that LIG3-targeting siRNAs were shown to knock down LIG3 mRNA levels to less than 50% of wild type levels, as indicated in FIG. 5.

### Example 20

### LIG3 Tissue Distribution

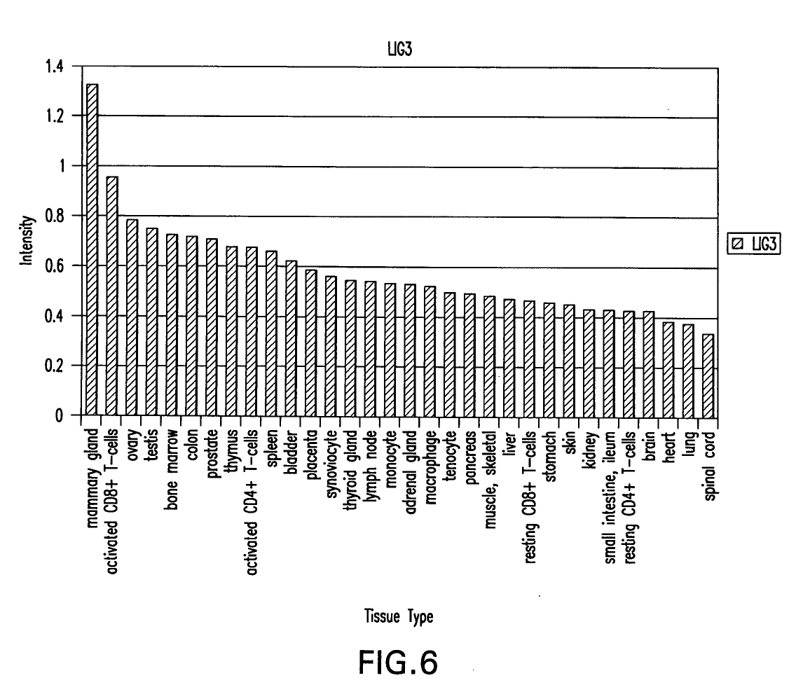

siRNAs chosen for further analysis were examined for expression in cells infected by HIV or tissues that harbor the virus, including CD4+ T-lymphocytes, macrophage, lymph node and thymus using Merck's proprietary Body Atlas, which contains data from microarray experiments carried out with many different tissues compared against a species-specific reference pool. LIG3 was found to be expressed in most tissues, with highest expression in mammary gland, as seen in FIG. 6.

### Example 21

### Effect of LIG3 on Tat-Mediated LTR Transactivation

The effect of LIG3 siRNAs on tat-mediated LTR transactivation was assayed by testing the effect of the siRNAs on expression of the LTR-β-galactosidase reporter gene in HeLa P4/R5 cells following transfection of a tat expression vector. The experiment was carried out as follows:

Day 1: HeLa (P4/R5) cells were plated at 3000 cells per well into 96-well Falcon

Plates.

Day 2: Cells were transfected with siRNAs as follows:

1. siRNAs were transfected at a final concentration of 100 nM using Oligofectamine (Invitrogen) at a final concentration of 0.5%. Positive and negative control siRNAs were the same as in Example 9; LIG3 siRNA were siRNAs 1, 2, and 3 from example 18.

2. 66 μL of Optimem/well was dispensed into a sterile 96-well plate, leaving the 12^{th} column empty.

3. 2 μL of siRNA (resuspended at 10 μM) from each well of the siRNA stock plate was transferred into the Optimem-containing plates such that the siRNA from well A3 of the mother plate is transferred into well A2 of the daughter plate, i.e., 2 μL of siRNA from each well is transferred into the corresponding plate into the same row position and the N-1 column position.

4. The resultant solution was mixed by pipetting up and down.

5. 240 μL Oligofectamine and 1210 μL Optimem were added to a microfuge Tube, which incubated 5 minutes at room temperature.

6. 12 μL of the oligofectamine was dispensed to each well and mixed by pipetting up and down. The plate was incubated at room temperature for 15 minutes.

7. 20 μL of the siRNA-oligofectamine complex was added to each well of the plate containing the HeLa (P4/R5) cells.

Day 4: Transfected HeLa(P4/R5) cells were transfected with pUCd5-Tat, and HIV 1-tat expression vector. The transfection mixture was prepared in bulk for the entire plate as follows:
1. Mix 1 was prepared (one plate, 100 samples), which consisted of:

| | |
|---|---|
| a. Lipofectamine 2000 | 50 μL |
| b. Opti-MEM | 2500 μL |

which was incubated at room temperature for 5 minutes.
2. Mix 2 was prepared (one plate, 100 samples), which consisted of:

| | |
|---|---|
| a. Opti-MEM | 2500 μL |
| b. PUC-D5 Tat (1 ng/μL) | 10 μL |

which was incubated at room temperature for 5 minutes.
3. Mix 3 was prepared, which combined Mix 1 and Mix 2 in their entirety, and was then incubated at room temperature for 20 minutes.
   4. For the transfection step, media was removed from the wells, 50 μL of Mix 3 and 50 μL of culture media was added to each well and incubated for 24 hrs.
Day 5: Beta-galactosidase activity (an indication of viral infection) was measured as follows:
   1. Media was removed from the cells.
   2. Cells were washed with 40 μL PBS per well.
   3. 20 μL of PBS was added to each well.
   4. 20 μL of Lysis buffer plus substrate (25:1) (Applied Biosystems, Cat# GSY10,000) were then added to each well and the plates were incubated at room temperature in the dark for 30 minutes.
   5. The plates were read using a VictorLight luminometer (PerkinElmer)
      LIG3 siRNAs were assayed in triplicate and found to reduce Tat-mediated transcription by 46%.

### Example 22

### Rescue of LIG3 siRNA-Mediated Inhibition of HIV Replication with expression of a non-targeted LIG3 cDNA

Day 1: HeLa (P4/R5) cells were plated at 3000 cells per well into 96-well Falcon plates.

Day 2: HeLa (P4/R5) cells were co-transfected with siRNA against the 3′UTR and cDNA of the tested gene as follows:
1. siRNAs targeting the 3′UTR of LIG3 were co-transfected with cDNA expressing LIG3 that did not include the 3′UTR. cDNA3.1 N-V5 DEST vector (Invitrogen) was included as a negative control for the same amount of LIG3 in the same vector. The ultimate LIG3 ORF clone was obtained from Invitrogen (IOH5124). Transfections were carried out in triplicate with Lipofectamine 2000 (Invitrogen) at a final concentration of 1%. siRNA final concentration is 50 nM, plasmid DNA is 100 ng/well.
LIG3 3′UTR siRNAs were:

| LIG3 siRNA #7: | |
|---|---|
| GGCAGAUAGACACAGUAUAdTdT | (SEQ ID NO: 39) |
| LIG3 siRNA #8 | |
| CAUACUCUCCUUUACCAUAdTdT | (SEQ ID NO: 40) |
| LIG3 siRNA #9: | |
| CCUUUACCAUACUACUGGAdTdT | (SEQ ID NO: 41) |

For each transfection, the following steps were carried out:
2. 1 μL Lipofectamine 2000 was mixed with 244 of Opti-MEM and incubated at room temperature for 5 minutes.
3. 23.5 μL of Opti-MEM was mixed with 0.5 μL of 10 uM siRNA and 1 μl of 100 ng/μl cDNA and incubated at room temperature for 5 minutes.
4. The mixture from step 2 and step 3 was combined and incubated at room temperature for 20 minutes.
5. 50 μL of the complex from step 4 was transferred to cells with 504 of culture media (DMEM+10% serum) to a final volume of 100 μL and incubated at 37° C. for 6 hours.
6. The media with transfection complex was removed and changed to fresh culture media and incubated at 37° C. overnight.

Day 3: The transfected HeLa(P41R5) cells were infected with HXB2 HIV as follows:
1. Media was removed from the cells
2. HXB2 HIV was diluted with media 400×. 100 μL of diluted HXB2 was added to each well.
3. Viral infection was allowed to proceed for 48 h.

Day 5: Beta-galactosidase activity was measured as follows:
1. 100 μL of Lysis buffer plus substrate (25:1)(Applied Biosystems, Cat# GSY10,000) was added to each well and the plates were incubated at room temperature in the dark for 30 minutes.
2. The plates were read using a VictorLight luminometer (PerkinElmer).

Two out of three siRNAs targeting the 3′UTR of LIG3 inhibited HIV infection to between 25 and 57% of levels after transfection of a nonsilencing siRNA. Co-transfection of these siRNAs with an expression vector for LIG3 cDNA lacking the 3′UTR (so the expressed cDNA can not be silenced by 3′UTR-targeting siRNAs) resulted in recovery of HIV infectivity in the cells. The degree of recovery ranged from 65-88% infectivity relative to controls in which cells were transfected with a nonsilencing siRNA (see FIG. 7). The difference between siRNA+vector-transfected and siRNA+cDNA transfected was statistically significant, with p<0.01 for siRNAs 7 and 9 (**), and p<0.05 for siRNA 8 (*).

### Example 23

### POLB siRNAs Inhibit HIV Infection

POLB is a DNA polymerase associated with the base excision repair pathway. The following experiments were carried out to determine whether transfection of siRNAs targeting POLB decreases HIV infection.

The following procedures were carried out:

Day 1: HeLa (P4/R5) cells were plated at 2000 cells per well in 4×96-well plates.

Day 2: HeLa (P4/R5) cells were transfected with siRNA pools as follows:
1) siRNAs were transfected at a final concentration of 100 nM using Oligofectamine (Invitrogen) at a final concentration of 0.5%. Positive and negative control siRNAs are included as follows:
   CDK9 (positive control): GUGGUCAACUUGAUUGAGAdTdT (SEQ ID NO: 23)
   Cyclin T1 (positive control): (purchased from Santa Cruz Biotechnology (cat #sc-35144) and luciferase, which was used as a negative control as in Example 1.

| POLB siRNA #1: | |
|---|---|
| GAGUGGAGCUGAAGCUAAGTT | (SEQ ID NO: 42) |
| POLB siRNA #2: | |
| CACUAGAAGAUCUCAGAAATT | (SEQ ID NO: 43) |
| POLB siRNA #3: | |
| CUCGUGAAGAGAUGUUACATT | (SEQ ID NO: 44) |
| POLB siRNA #4: | |
| CUAUUUCACUGGGAGUGAUTT | (SEQ ID NO: 45) |
| POLB siRNA #5: | |
| GCGAAUUGGGCUGAAAUAUTT | (SEQ ID NO: 46) |
| POLB siRNA #6: | |
| GGUUGAUACCCAAAGAUCATT | (SEQ ID NO: 47) |

15. 66 μL of Optimem/well was dispensed into a sterile 96-well plate, leaving the 12^{th} column empty.

16. 2 μL of siRNA (resuspended at 10 μM) was transferred from each well of the siRNA stock plate into the Optimem-containing plates, such that the siRNA from well A3 of the mother plate is transferred into well A2 of the daughter plate.

17. The resultant solution was mixed by pipetting up and down.

18. 240 μL Oligofectamine and 1210 μL Optimem were then added to a microfuge tube, which was incubated 5 minutes at room temperature.

19. 12 μL of the oligofectamine was dispensed to each well and mixed by pipetting up and down. The plate was then incubated at room temperature for 15 minutes.

20. 20 μL of the siRNA-oligofectamine complex was added to each well of the HeLa(P4/R5) cells.

Day 3: Transfected HeLa(P4/R5) cells were infected with HXB2 HIV in the presence and absence of an integrase inhibitor as follows:
1. Media was removed from the cells
2. 80 μL fresh media was added to each well
3. An integrase inhibitor was diluted to 20 nM in media. 404 of the 20 nM integrase inhibitor solution was added to each well of two of the plates (the final concentration of the integrase inhibitor was equal to the IC50 of the compound for inhibition of viral infection in this assay (Anthony et al, 2004). Forty μL of media without compound was added to the remaining two plates.
4. HXB2 HIV was diluted with media 100× and forty μL of diluted HXB2 was added to each well.
5. Viral infection was allowed to proceed for 48 h.

Day 5. Beta-galactosidase activity was measured as follows:
1. Media was removed from the cells.
2. Cells were washed with 200 μL PBS per well.
3. 20 μL Lysis Buffer (Galacto-Light Plus, Tropix, cat# BL100P) containing DTT was added to each well, and the plates were shaken for 10 minutes.
4. 80 μL of substrate was then added to each well and the plates were incubated at room temperature in the dark for 1 hour.
5. 1004 of enhancing solution was added to each well and the plates were read using a Dynex luminometer.

Readings for each plate were normalized to the reading for the luciferase negative control and expressed as “% of Luciferase Control”. Hits were considered to be those siRNA pools that suppressed beta-galactosidase activity by 40% or more, or those that showed 30% or greater inhibition of beta-galactosidase activity in the presence of IC50 levels of the integrase inhibitor compared to the absence of compound treatment. It was found that three out of the six siRNAs tested (siRNA #s 2, 4, and 5) were found to decrease HIV infectivity by more than 40%.

### Example 24

### POLB siRNAs are not Cytotoxic

The LIG3 siRNA pool was examined for cytotoxic effects in the cytotoxicity assay described in Example 23. siRNAs that led to viability levels of less than 70% as determined by Alamar Blue fluorescence were considered to be cytotoxic. POLB siRNAs did not show any evidence of cytotoxicity following transfection into HeLa P4/R5 cells.

### Example 25

### POLB Tissue Distribution

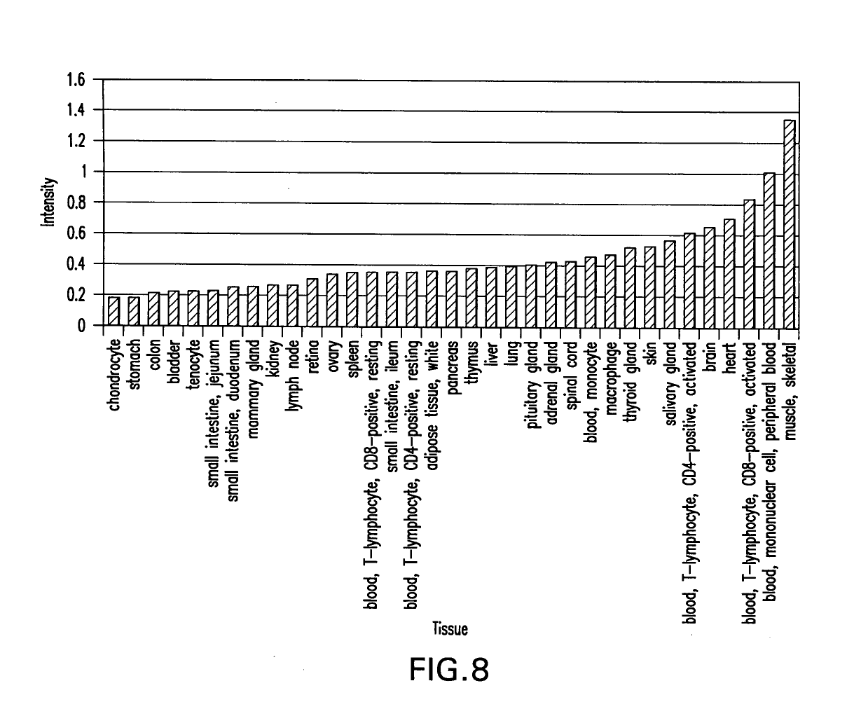

siRNAs chosen for further analysis were examined for expression in cells infected by HIV or tissues that harbor the virus, including CD4+ T-lymphocytes, macrophage, lymph node and thymus using Merck's proprietary Body Atlas, which contains data from microarray experiments carried out with many different tissues compared against a species-specific reference pool. POLB is expressed in most tissues with highest expression in skeletal muscle, as seen in FIG. 8.

### Example 26

### Effect of POLB on Tat-Mediated LTR Transactivation

The effect of POLB siRNAs on tat-mediated LTR transactivation was assayed by testing the effect of the siRNAs on expression of the LTR-β-galactosidase reporter gene in HeLa P4/R5 cells following transfection of a tat expression vector. The experiment was carried out as follows:

Day 1: HeLa (P4/R5) cells were plated at 3000 cells per well into 96-well Falcon plates

Day 2: Cells were transfected with siRNAs as follows:

2. siRNAs were transfected at a final concentration of 100 nM using Oligofectamine (Invitrogen) at a final concentration of 0.5%. Positive and negative control siRNAs were the same as in Example 9; POLB siRNA were siRNAs 1, 2, and 3 from example 23.

2. 66 μL of Optimem/well was dispensed into a sterile 96-well plate, leaving the 12^{th} column empty.

3. 2 μL of siRNA (resuspended at 10 μM) from each well of the siRNA stock plate was transferred into the Optimem-containing plates such that the siRNA from well A3 of the mother plate is transferred into well A2 of the daughter plate (2 μL of siRNA from each well is transferred into the corresponding plate into the same row position and the N-1 column position).

4. The resultant solution was mixed by pipetting up and down.

5. 240 μL Oligofectamine and 12104 Optimem were added to a microfuge tube which incubated 5 minutes at room temperature.

6. 12 μL of the oligofectamine was dispensed to each well and mixed by pipetting up and down. The plate was incubated at room temperature for 15 minutes.

7. 20 μL of the siRNA-oligofectamine complex was added to each well of the plate containing the HeLa (P4/R5) cells.

Day 4: Transfected HeLa(P4/R5) cells were transfected with pUCd5-Tat, and HIV1-tat expression vector. The transfection mixture was prepared in bulk for the entire plate as follows:
1. Mix 1 was prepared (one plate, 100 samples), which consisted of:

| | |
|---|---|
| c. Lipofectamine 2000 | 50 μL |
| d. Opti-MEM | 2500 μL |

which was incubated at room temperature for 5 minutes.
2. Mix 2 was prepared (one plate, 100 samples), which consisted of:

| | |
|---|---|
| c. Opti-MEM | 2500 μL |
| d. PUC-D5 Tat (1 ng/μL) | 10 μL |

which was incubated at room temperature for 5 minutes.
3. Mix 3 was prepared, which combined Mix 1 and Mix 2 in their entirety, and was then incubated at room temperature for 20 minutes.
5. For the transfection step, media was removed from the wells, 50 μL of Mix 3 and 50 μL of culture media was added to each well and incubated for 24 hrs.

Day 5: Beta-galactosidase activity (an indication of viral infection) was measured as follows:
1. Media was removed from the cells.
2. Cells were washed with 40 μL PBS per well.
3. 20 μL of PBS was added to each well.
4. 20 μL of Lysis buffer plus substrate (25:1) (Applied Biosystems, Cat#GSY10,000) were then added to each well and the plates were incubated at room temperature in the dark for 30 minutes.
5. The plates were read using a VictorLight luminometer (PerkinElmer) POLB siRNAs were assayed in triplicate and found to have no substantial effect on tat-mediated transactivation.

### Example 27

### XRCC1 siRNAs Inhibit HIV Infection

XRCC1 is a DNA repair protein also associated with the base excision repair pathway. The following experiments were carried out to determine whether transfection of siRNAs targeting XRCC1 decreases HIV infection.

The following procedures were carried out:

Day 1: HeLa (P4/R5) cells were plated at 2000 cells per well in 4×96-well plates.

Day 2: HeLa (P4/R5) cells were transfected with siRNA pools as follows:
1) siRNAs were transfected at a final concentration of 100 nM using Oligofectamine (Invitrogen) at a final concentration of 0.5%. Positive and negative control siRNAs are included as follows:
CDK9 (positive control): GUGGUCAACUUGAUUGAGAdTdT (SEQ ID NO: 23)
Cyclin T1 (positive control): (purchased from Santa Cruz Biotechnology (cat #sc-35144) and luciferase, which was used as a negative control as in Example 1.

| XRCC1 siRNA #1: | |
|---|---|
| CUGUUCCCAAGAGACCUAATT | (SEQ ID NO: 48) |
| XRCC1 siRNA #2: | |
| GUCCUUCUGGUCACCUCAUTT | (SEQ ID NO: 49) |
| XRCC1 siRNA #3: | |
| GCUCCGAGCUGCGAGAUAATT | (SEQ ID NO: 50) |
| XRCC1 siRNA #4: | |
| GCAAGCACUUCUUUCUUUATT | (SEQ ID NO: 51) |
| XRCC1 siRNA #5: | |
| CGAUACGUCACAGCCUUCATT | (SEQ ID NO: 52) |
| XRCC1 siRNA #6: | |
| CAGUCAGAAGGACAGGACATT | (SEQ ID NO: 53) |

21. 66 μL of Optimem/well was dispensed into a sterile 96-well plate, leaving the 12^{th} column empty.

22. 2 μL of siRNA (resuspended at 10 μM) was transferred from each well of the siRNA stock plate into the Optimem-containing plates, such that the siRNA from well A3 of the mother plate is transferred into well A2 of the daughter plate (2 μL of siRNA from each well is transferred into the corresponding plate into the same row position and the N-1 column position).

23. The resultant solution was mixed by pipetting up and down.

24. 240 μL Oligofectamine and 1210 μL Optimem were then added to a microfuge tube, which was incubated 5 minutes at room temperature.

25. 12 μL of the oligofectamine was dispensed to each well and mixed by pipetting up and down. The plate was then incubated at room temperature for 15 minutes.

26. 20 μL of the siRNA-oligofectamine complex was added to each well of the HeLa(P4/R5) cells.

Day 3: Transfected HeLa(P4/R5) cells were infected with HXB2 HIV in the presence and absence of an integrase inhibitor as follows:
6. Media was removed from the cells 7. 80 μL fresh media was added to each well
8. An integrase inhibitor was diluted to 20 nM in media. 40 μL of the 20 nM integrase inhibitor solution was added to each well of two of the plates (the final concentration of the integrase inhibitor was equal to the IC50 of the compound for inhibition of viral infection in this assay (Anthony et al, 2004). Forty μL of media without compound was added to the remaining two plates.
9. HXB2 HIV was diluted with media 100× and fortyl μL of diluted HXB2 was added to each well.
10. Viral infection was allowed to proceed for 48 h.

Day 5. Beta-galactosidase activity was measured as follows:
6. Media was removed from the cells.
7. Cells were washed with 200 μL PBS per well.
8. 20 μL Lysis Buffer (Galacto-Light Plus, Tropix, cat# BL100P) containing DTT was added to each well, and the plates were shaken for 10 minutes.
9. 80 μL of substrate was then added to each well and the plates were incubated at room temperature in the dark for 1 hour.
10. 100 μL of enhancing solution was added to each well and the plates were read using a Dynex luminometer.

Readings for each plate were normalized to the reading for the luciferase negative control and expressed as “% of Luciferase Control”. Hits were considered to be those siRNA pools that suppressed beta-galactosidase activity by 40% or more, or those that showed 30% or greater inhibition of beta-galactosidase activity in the presence of IC50 levels of the integrase inhibitor compared to the absence of compound treatment. It was found that two out of the six siRNAs tested (siRNA #s 2 and 4) were found to decrease HIV infectivity by more than 40%.

### Example 28

### XRCC1 siRNAs are not Cytotoxic

The LIG3 siRNA pool was also examined for cytotoxic effects in the cytotoxicity assay described in Example 23. siRNAs that led to viability levels of less than 70% as determined by Alamar Blue fluorescence were considered to be cytotoxic. XRCC1 siRNAs did not show any evidence of cytotoxicity following transfection into HeLa P4/R5 cells.

### Example 29

### XRCC1 Tissue Distribution

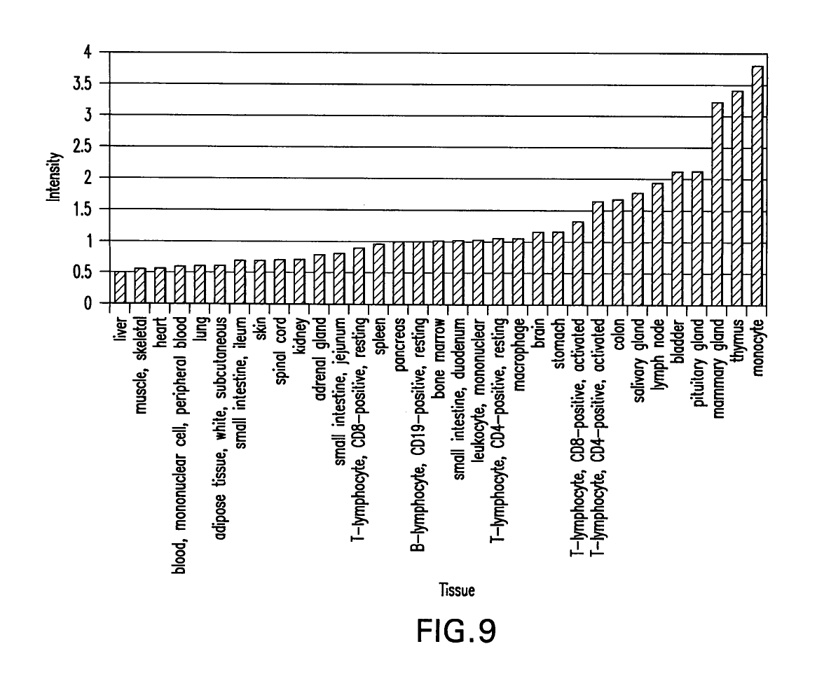

siRNAs chosen for further analysis were examined for expression in cells infected by HIV or tissues that harbor the virus, including CD4+ T-lymphocytes, macrophage, lymph node and thymus using Merck's proprietary Body Atlas, which contains data from microarray experiments carried out with many different tissues compared against a species-specific reference pool. XRCC1 is expressed in most tissues, with highest expression in monocytes and thymus, as seen in FIG. 9.

### Example 30

### Effect of XRCC1 on Tat-Mediated LTR Transactivation

The effect of XRCC1 siRNAs on tat-mediated LTR transactivation was assayed by testing the effect of the siRNAs on expression of the LTR-β-galactosidase reporter gene in HeLa P4/R5 cells following transfection of a tat expression vector. The experiment was carried out as follows:

Day 1: HeLa (P4/R5) cells were plated at 3000 cells per well into 96-well Falcon plates.

Day 2: Cells were transfected with siRNAs as follows:

3. siRNAs were transfected at a final concentration of 100 nM using Oligofectamine (Invitrogen) at a final concentration of 0.5%. Positive and negative control siRNAs were the same as in Example 9; XRCC1 siRNAs used were siRNAs 1, 2, and 3 from example 27.

2. 66 μL of Optimem/well was dispensed into a sterile 96-well plate, leaving the 12^{th} column empty.

3. 2 μL of siRNA (resuspended at 10 μM) from each well of the siRNA stock plate was transferred into the Optimem-containing plates such that the siRNA from well A3 of the mother plate is transferred into well A2 of the daughter plate (2 μL of siRNA from each well is transferred into the corresponding plate into the same row position and the N-1 column position).

4. The resultant solution was mixed by pipetting up and down.

5. 240 μL Oligofectamine and 1210 μL Optimem were added to a microfuge tube which incubated 5 minutes at room temperature.

6. 12 μL of the oligofectamine was dispensed to each well and mixed by pipetting up and down. The plate was incubated at room temperature for 15 minutes.

7. 20 μL of the siRNA-oligofectamine complex was added to each well of the plate containing the HeLa (P4/R5) cells.

Day 4: Transfected HeLa(P4/R5) cells were transfected with pUCd5-Tat, and HIV 1-tat expression vector. The transfection mixture was prepared in bulk for the entire plate as follows:
1. Mix 1 was prepared (one plate, 100 samples), which consisted of:

| | |
|---|---|
| a. Lipofectamine 2000 | 50 μL |
| b. Opti-MEM | 2500 μL |

which was incubated at room temperature for 5 minutes.
2. Mix 2 was prepared (one plate, 100 samples), which consisted of:

| | |
|---|---|
| a. Opti-MEM | 2500 μL |
| b. PUC-D5 Tat (1 ng/μL) | 10 μL |

which was incubated at room temperature for 5 minutes.
3. Mix 3 was prepared, which combined Mix 1 and Mix 2 in their entirety, and was then incubated at room temperature for 20 minutes.
4. For the transfection step, media was removed from the wells, 50 μL of Mix 3 and 50 μL of culture media was added to each well and incubated for 24 hrs.
Day 5: Beta-galactosidase activity (an indication of viral infection) was measured as follows:
   1. Media was removed from the cells.
   2. Cells were washed with 40 μL PBS per well.
   3. 20 μL of PBS was added to each well.
   4. 20 μL of Lysis buffer plus substrate (25:1) (Applied Biosystems, Cat# GSY10,000) were then added to each well and the plates were incubated at room temperature in the dark for 30 minutes.
   5. The plates were read using a VictorLight luminometer (PerkinElmer)

XRCC1 siRNAs were assayed in triplicate and found to have no substantial effect on tat-mediated transactivation.

Other embodiments are within the scope of the following claims. All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. All such variations apparent to those skilled in the art are deemed to be within the spirit, scope and concept of the invention as defined by the appended claims.

1. (canceled)

2. An siNA that inhibits or downregulates expression of one or more genes involved in DNA repair, said genes selected from the group consisting of mutyh; neil3; lig3; polB; and xrcc1 and genes substantially similar thereto.

3. An assay for identifying a compound as an HIV inhibitor comprising the steps of:
(a) identifying a compound that downregulates or inhibits the activity or expression of a target protein that is a component of a DNA repair pathway of a human cell; and
(b) determining the ability of said compound to inhibit HIV.

4. The assay of claim 3, wherein the target protein is selected from the group consisting of: MUTYH; NEIL3; LIG3; POLB; and XRCC1 and proteins substantially similar thereto.

5. A method of screening for a compound which inhibits or downregulates the expression or activity of one or more components of a base excision DNA repair pathway of a human cell, thereby decreasing HIV infection, comprising the steps of:
(a) contacting the one or more components of a base excision DNA repair pathway of a human cell with a noncircularized HIV DNA in the presence or absence of a test compound;
and
(b) measuring the amount of HIV DNA circularization,
wherein decreased circularization in the presence of the test compounds as compared to the amount of circularization in the absence of said test compound indicates that the test compound inhibits or downregulates the expression or activity of one or more components of a base excision DNA repair pathway.

6. The method of claim 5, wherein the one or more components of the base excision DNA repair pathway of a human cell is a nucleic acid molecule encoding a polypeptide selected from the group consisting of MUTYH; NEIL3; LIG3; POLB; and XRCC1; and proteins substantially similar thereto.

7. The assay of claim 3 wherein the DNA repair pathway is base excision repair.

8. The method of claim 5, wherein the one or more components of the base excision DNA repair pathway of a human cell is a polypeptide selected from the group consisting of MUTYH; NEIL3; LIG3; POLB; and XRCC1; and proteins substantially similar thereto.
